SEQUENCE LISTING (7 sequences)

SEQ ID NO: 1 - protein, 241 aa, Homo sapiens
MSNEVETSATNGQPDQQAAPKAPSKKEKKKGPEKTDEYLLARFKGDGVKYKAKLIGIDDVPDARGDKMSQDSMMKLKGMAARGRSQGQHKQRIWVNISLSGIKIIDEKTGVIEHEHPVNKISFIARDVTDNRAFGYVCGGEGQHQFFAIKTGQQAEPLVVDLKDLFQVIYNVKKKEEEKKKIEEASKAVENGSEALMILDDQTNKLKSGVDQMDLFGDMSTPPDLNSPTESKDILLVDLNS

SEQ ID NO: 2 - protein, 8 aa, Homo sapiens
SKKEKKAG

SEQ ID NO: 3 - protein, 8 aa, Homo sapiens
SKKEKKAA

SEQ ID NO: 4 - protein, 8 aa, Homo sapiens
SKKEKKKA

SEQ ID NO: 5 - protein, 17 aa, Homo sapiens
PEKTDEYLLARFAGDGV

SEQ ID NO: 6 - protein, 17 aa, Homo sapiens
PEKTDEYLLARFAADGV

SEQ ID NO: 7 - protein, 17 aa, Homo sapiens
PEKTDEYLLARFKADGV

(19) United States
(12) Patent Application Publication
(10) Pub. No.: US 2010/0285583 A1
(43) Pub. Date: Nov. 11, 2010

(54) COMPOUNDS AND METHODS FOR INHIBITING PLATELET AGGREGATION

(76) Inventors: Daniel G.S. Capelluto, Blacksburg, VA (US); Carla V. Finkielstein, Blacksburg, VA (US); Karen E. Drahos, Fort Wayne, IN (US); John D. Welsh, Pennington, NJ (US)

Correspondence Address: BLANK ROME LLP, WATERGATE, 600 NEW HAMPSHIRE AVENUE, N.W., WASHINGTON, DC, 20037, US

(21) Appl. No.: 12/775,834

(22) Filed: May 7, 2010

Related U.S. Application Data
(60) Provisional application No. 61/176,583, filed on May 8, 2009.

(51) Int. Cl. C12N 5/078; C07K 7/00; C07K 7/06; C07K 7/08; C07K 14/47; C07H 21/00; C12N 1/21

(52) U.S. Cl. 435/375; 530/327; 530/329; 530/328; 530/330; 530/326; 530/350; 536/23.5; 435/252.33

(57) ABSTRACT

The present invention relates to compounds and methods for inhibiting platelet aggregation. In an embodiment, the compound of the present invention contains the sulfatide binding region of the N-terminal phosphotyrosine binding domain (N-PTB) of Disabled-2 (Dab2).

This application claims the priority of U.S. Provisional Patent Application No. 61/176,583, filed May 8, 2009, which is incorporated herein by reference.

### FIELD OF THE INVENTION

The present invention relates to compounds and methods for inhibiting platelet aggregation. In particular, the compound of the present invention contains at least one sufatide binding polypeptide, preferably at least one sulfatide binding region of the N-terminal phosphotyrosine binding (N-PTB or PTB) domain of the Disabled-2 (Dab2) protein.

### BACKGROUND OF THE INVENTION

Thrombotic complications are a major cause of death in the industrialized world. Examples of these complications include acute myocardial infarction, unstable angina, chronic stable angina, transient ischemic attacks, strokes, peripheral vascular disease, preeclampsia/eclampsia, deep venous thrombosis, embolism, disseminated intravascular coagulation and thrombotic cytopenic purpura. Thrombotic and restenotic complications also occur following invasive procedures, e.g., angioplasty, carotid endarterectomy, post CABG (coronary artery bypass graft) surgery, vascular graft surgery, stent placements and insertion of endovascular devices and protheses. It is generally thought that platelet aggregates play a critical role in these events. Blood platelets, which normally circulate freely in the vasculature, become activated and aggregate to form a thrombus with disturbed blood flow caused by ruptured atherosclerotic lesions or by invasive treatments such as angioplasty, resulting in vascular occlusion. Platelet activation can be initiated by a variety of agents, e.g., exposed subendothelial matrix molecules such as collagen, or by thrombin, which is formed in the coagulation cascade.

A plethora of drugs for anti-platelet therapy are available for treatment of bleeding disorders and pathological thrombosis, including cyclooxygenase inhibitors (such as aspirin), ADP-receptor inhibitors (such as clopidogrel), and α_{IIb}β₃ integrin inhibitors (such as abciximab). The success of treatments that intervene during acute thrombosis depends on the timing of their application. However, treatment of patients with cardiovascular disease using exogenous compounds has undesirable side effects such as stomach ulcers, increased bleeding, and activation of platelets via the receptors they target, such as the α_{IIb}β₃ integrin receptor. The side effects are generally due to the fact that the compounds have long half-lives in the blood stream, which allows the compounds to assert their effects long after the treatment is needed. Therefore, there remains a need for anti-platelet compounds having a shorter half-life so that the adverse side effects can be eliminated.

### SUMMARY OF THE INVENTION

The present inventors have discovered that two pools of Disabled-2 (Dab2) are present on the surface of activated platelets. The first pool binds to the αIIβ3 integrin receptors forming Dab2/α_{IIb}β₃ integrin receptor complexes. The second pool binds to sulfatides forming Dab2-sulfatide complexes. Moreover, the inventors have identified the polybasic region within Dab2 N-PTB responsible sulfatide binding. The first pool negatively controls platelet aggregation by competing with fibrinogen for binding to the α_{IIb}β₃ integrin receptor. The second pool binds to sulfatides at the platelet surface rendering Dab2 inaccessible for thrombin cleavage and preventing the association of pro-coagulant proteins to sulfatides.

In an embodiment, the present invention relates to compounds for binding sulfatides. By binding sulfatides, the compound inhibits platelet aggregation by shifting the surface Dab2 in favor of the first pool (Dab2-α_{IIb}β₃ integrin receptor complexes). Preferably, the compounds for binding sulfatides contain both sulfatide binding domains within N-PTB. More preferably, the compounds contain amino acids 24-31 of SEQ ID NO: 1 and/or amino acids 49-54 of SEQ ID NO: 1. A compound containing a homolog of the N-PTB domain is also appropriate as long the homolog is still able to bind sulfatides.

In another embodiment, the present invention relates to a method for inhibiting platelet aggregation. This method involves contacting the compound for sulfatides binding to platelets, preferably activated platelets. The compound, thus, competes with Dab2 on the surface of the platelet for sulfatides, thereby resulting in inhibition of platelet-platelet and platelet-leukocyte bindings, and thus, platelet aggregation and clot stabilization.

In another embodiment, the present invention relates to a method for inhibiting platelet-leukocyte binding by contacting the compound for sulfatide binding to platelets, preferably activated platelets. Upon contacting with the platelets, the compound binds sulfatides, thereby preventing secondary degranulation of α-granules and release of P-selectin, resulting in inhibition of platelet-leukocyte binding.

In yet another embodiment, the present invention relates to a method for activating platelet aggregation. This method involves contacting sulfatides with platelets to shift the surface Dab2 in favor of the second pool (Dab2- sulfatide complexes), thereby, stimulating platelet aggregation.

### BRIEF DESCRIPTION OF THE DRAWINGS

FIG. 1. The Dab2 PTB domain interacts with sulfatides. (A) Sequence alignment of the proposed regions of the N-PTB domain involved in both sulfatide and PtdIns(4,5)P₂ ligation (bold residues). Amino acids that interact with sulfatides or PtdIns(4,5)P₂ and are boxed. Consensus motifs for sulfatide binding are indicated at the bottom. (B) Nitrocellulose membranes (Sphingolipid strips) containing the indicated lipids were probed with 0.2 μg/ml GST-Dab2 PTB, according to the manufacturer's instructions. (C) Liposome binding assay of the Dab2 PTB domain and its mutants with liposomes in the absence and presence of sulfatides. Lanes labeled with ‘S’ and ‘P’ represent proteins present in supernatants and pellets after centrifugation. GST was used as a negative control. (D) Same as C but in the absence and presence of PtdIns(4,5)P₂.

FIG. 2. Kinetic and competitive analyses of the N-PTB lipid ligands. (A) The interactions of Dab2 PTB with immobilized sulfatide (left) and PtdIns(4,5)P₂ (right) liposomes were analyzed by SPR detection. Resonance units indicating the bound protein fraction at increasing protein concentrations were plotted. (B) Nitrocellulose filters containing increasing amounts of sulfatides where incubated with either free or PtdIns(4,5)P2-bound GST-Dab2 N-PTB domain. Quantification of the binding is shown on the right. (C) Competition of the lipids analyzed by SPR detection. Immobilized sulfatide liposomes were exposed to 5 μM Dab2 PTB (top) and PTB^{4M} (bottom) with increasing concentrations of PtdIns(4,5)P₂ pre-incubated with the protein.

FIG. 3. Sulfatides protect N-PTB from thrombin proteolysis. (A) Ribbon (top) and surface (bottom) representation of the N-PTB domain. Residues engaged in sulfatide ligation are indicated in red and yellow respectively. Lys53, a residue critical for recognition of both lipids, is labeled in orange. (B) The Dab2 PTB domain was incubated in the absence and presence of thrombin at the indicated time points and analyzed by SDS-PAGE. (C) The Dab2 PTB domain was pre-incubated with liposomes without (top) and with (bottom) sulfatides and incubated with thrombin as described in A. (D) The N-PTB domain was pre-incubated with liposomes without (top) and with (bottom) PtdIns(4,5)P₂ and proceeded as described in A.

FIG. 4. Roles of sulfatides and PtdIns(4,5)P₂ in N-PTB subcellular localization. Human washed platelets were incubated with Dab2 PTB, PTB^{4M} or PTB^{K53K90} domains (1.9 μM each) for 5 min at room temperature in the presence of 0.25 g/L fibrinogen (left panels). Endogenous Dab2 was also followed by the same procedure. Aggregation was initiated by the addition of TRAP at room temperature. Samples were fixed at 3 min (center panels) and 10 min (right panels) and subcellular localization of the proteins was visualized using anti-Dab2 and Cy3-coupled secondary antibodies. Quantification of the percentage of platelets showing binding and internalization of both Dab2 PTB and PTB^{K53K90} domains are represented by diagram bars. Scale bar is 5 μm.

FIG. 5. Two pools of Dab2 likely exist at the activated platelet surface. (A) Human washed platelets were activated in fibrinogen-coated wells in the presence of N-PTB or PTB^{4M} proteins (1.9 μM), fixed, and stained with Wright stain. Stain was eluted with 20% ethanol and quantified at 415 nm. (B) Model showing two pools of Dab2 at the activated platelet surface (integrin receptor bound protein and sulfatide bound protein) and the PtdIns(4,5)P₂ mediated endocytosis of Dab2.

FIG. 6. N-PTB-sulfatide interaction requires residues from both conserved basic motifs. (A) Nitrocellulose membranes containing the indicated pmoles of sulfatides were probed with 1 μg/ml GST or GST-PTB constructs. (B) Liposome binding assay of Dab2 PTB mutants in the absence or presence of sulfatides. Lanes labeled ‘S’ and ‘P’ represent proteins present in supernatants and pellets after centrifugation.

FIG. 7. Mutations do not alter the secondary structure of Dab2 PTB. Circular dichroism (CD) was performed with 5 μM PTB constructs. Spectra were converted to mean residue ellipticity using DICHROWEB and deconvoluted using CDSSTR.

FIG. 8. Mutations in the sulfatide binding sites do not significantly alter PtdIns(4,5)P₂binding. (A) Nitrocellulose membranes containing the indicated pmoles of PtdIns(4,5)P₂ were probed with 1 μg/ml GST or GST-PTB constructs. (B)Liposome binding assay of N-PTB mutants in the absence or presence of PtdIns(4,5)P₂. Lanes labeled ‘S’ and ‘P’ represent proteins in supernatant and pellet fractions after centrifugation.

FIG. 9. Sulfatide-phosphoinositide competition for N-PTB binding does not occur nonspecifically. GST-Dab2 PTB was pre-incubated in the absence and presence of 10-fold excess of PtdIns(3)P and further incubated with nitrocellulose membranes containing increasing amounts of sulfatides. GST was used as a negative control. Quantification of the binding is shown on the right.

FIG. 10. Controls for immunofluorescence analysis. Human washed platelets were incubated with Bovine Serum Albumin (BSA) control (1.9 μM), dimethyl sulfoxide (DMSO), or H₂O (vehicle controls) for 5 min at room temperature in the presence of 0.25 g/L fibrinogen (left panels). Aggregation was initiated by the addition of TRAP at room temperature. Samples were fixed at 3 min (center panels) and 10 min (right panels) and subcellular localization of the proteins was visualized using anti-Dab2 and Cy3-coupled secondary antibodies. Scale bar is 5 μm.

FIG. 11. Dab2 PTB^{D66E} shows little decrease in binding and internalization in activated platelets. Human washed platelets were incubated with Dab2 PTB^{D66E} for 5 min at room temperature in the presence of 0.25 g/L fibrinogen (left panel). Aggregation was initiated by the addition of TRAP at room temperature. Samples were fixed at 3 min (center panel) and 10 min (right panel) and subcellular localization of the proteins was visualized using anti-Dab2 and Cy3-coupled secondary antibodies. Scale bar is 5 μm.

FIG. 12. Sulfatide induced de-granulation. (A) P-selectin interaction with sulfatides on adjacent platelets stabilizes aggregates, as well as stimulating p38 mediated de-granulation leading to increased surface P-selectin and α_{IIb}β₃ integrin. (B) The affect of sulfatides on surface P-selectin and α_{IIb}β₃ levels. Platelets treated with ADP (30 μM) or TRAP (10 μM) were exposed to sulfatides (50 μg/mL) in the form of enriched liposomes, and changes in surface receptors were monitored using fluorescence signal detected by FACS. P-selectin marker was PE-anti-human CD62P and the α_{IIb}β₃ integrin marker was FITC-anti-human α_{IIb}β₃. The median fluorescence was measured as a representative point of the population. Each bar in the bar graph represents the average of three separate reactions. The experiment was done multiple times and this is a representative experiment.

FIG. 13. N-PTB inhibition of sulfatide induced de-granulation. Platelets were either left unactivated or stimulated with ADP (30 μM). ADP stimulated platelets were also treated with either sulfatide (50 μg/mL) enriched liposomes (sulfatides) or un-enriched liposomes (Control-Lipo). Platelets were stimulated with sulfatides in the presence of N-PTB or N-PTB4M. After incubation for 6 minutes the reaction was fixed and P-selectin marker was added. (A) Graph of the median fluorescence of P-selectin marker on the surface of platelets. Platelets are treated with ADP, either control or sulfatide liposomes, and different mutant constructs of N-PTB. (B and C) Representative chromatograms of the fluorescent signal detected for each reaction. Chromatograms show the fluorescent signal for each platelet detected and shifts in the peaks represent changes in the marker presence. (B) Black bars represent the shift of ADP stimulated platelets and control-lipo+ADP platelets. (C) A black bar represents the shift of sulfatide stimulated platelets in the presence of different N-PTB constructs. Sulfatide stimulation results in a shift right, while inhibition prevents any shift.

FIG. 14. N-PTB inhibition of sulfatide induced de-granulation. Platelets were either left unactivated or stimulated with ADP (30 μM). ADP stimulated platelets were also treated with either sulfatide (50 μg/mL) enriched liposomes (sulfatides) or un-enriched liposomes (Control-Lipo). Platelets were stimulated with sulfatides in the presence of N-PTB or N-PTB4M. After incubation for 6 minutes the reaction was fixed and α_{IIb}β₃ integrin marker was added. (A) Graph of the median fluorescence of P-selectin marker on the surface of platelets. Platelets are treated with ADP, either control or sulfatide liposomes, and different mutant constructs of N-PTB. (B and C) Representative chromatograms of the fluorescent signal detected for each reaction. Chromatograms show the fluorescent signal for each platelet detected and shifts in the peaks represent changes in the marker presence. (B) Black bars represent the shift of ADP stimulated platelets and control-lipo+ADP platelets. (C) A black bar represents the shift of sulfatide stimulated platelets in the presence of different N-PTB constructs. Sulfatide stimulation results in a shift right, while inhibition by N-PTB prevents any shift. FIG. 15. IC₅₀ curves of N-PTB and N-PTB^{D66E} titrations. Titration curves of increasing concentrations of (A) N-PTB and (B) N-PTB^{D66E}. Increasing amounts of N-PTB and N-PTB D66E (1 nM-1 μM) were incubated with platelets before the addition of sulfatide enriched liposomes as previously described. The resulting P-selectin marker signals were detected by FACS and plotted as a titration curve. Inhibition of sulfatides was calculated using sulfatide induced P-selectin marker expression, with complete inhibition being considered when P-selectin levels return to ADP stimulated platelets. Logarithmic curves were used to model the inhibition and IC₅₀ values were calculated using the formulas produced by the curves.

FIG. 16. Platelet Aggregation under flow assay. Platelets were either left untreated or incubated with either N-PTB or N-PTB4M. The platelets were then mixed with either sulfatide enriched liposomes, or un-enriched liposomes and immediately pumped through the microfluidics channel at a shear rate of 70 s⁻¹. The channel was coated with soluble adhesive proteins from human plasma. Platelet adhesion and aggregation was monitored using bright field microscopy, and pictures of the channel were taken at 30 sec, 3 min, and 10 min. Representative clots are shown.

FIG. 17. Platelet-leukocyte binding is mediated by sulfatides. (A) Sulfatides stimulate platelet P-selectin as well as stimulating leukocytes directly, Dab2 is able to inhibit platelet-leukocyte aggregation through sulfatide binding. (B) Platelet and leukocyte mixtures (10⁸ platelets/mL and 10⁷ leukocytes/mL) were incubated with either N-PTB or N-PTB4M. Liposomes, either sulfatide enriched or not, were added to the mixtures stimulated with ADP (30 μM). Reactions incubate for 6 minutes and are fixed and APC-anti-human CD42b is added. The CD42b fluorescence is quantified using FACS analysis. Leukocytes are identified based on their forward and side scatter plots as distinctive from platelets. Graph of the median fluorescence signal of platelet marker CD42b detected in the leukocyte population represents platelet-leukocyte interactions. (C and D) FACS chromatograms showing the fluorescence of CD42b within the leukocytes for each treatment, black bars represent the resulting shifts in platelet signal.

FIG. 18. Platelet-leukocyte aggregation under flow. Platelet-leukocyte mixtures (10⁸ platelets/mL and 10⁷ leukocytes/mL) were flown through a microfluidics channel. The channel was coated with adhesive proteins and the flow produced a shear rate of 70 s⁻¹. Platelet-leukocyte aggregates were monitored using bright field microscopy throughout the channel after 10 minutes of stead flow. (A) Untreated, as well as control and sulfatide liposome treated cell suspensions were flown through the channel for 10 minutes, and representative aggregate pictures are shown. (B) Platelet-leukocyte mixtures containing sulfatide liposomes and either N-PTB or N-PTB^{4M} (10 μM) were flown for 10 min, and representative aggregate pictures are shown.

FIG. 19. The role of Dab2 in platelet aggregation. Dab2 inhibits the α_{IIb}β₃ integrin both intracellularly and extracellularly. Dab2 binding of sulfatides inhibits platelet-sulfatide interactions which decreases clot stability and blocks de-granulation. Dab2 inhibition of de-granulation results in decreased P-selectin expression. Decreased P-selectin and blocking sulfatides from stimulating leukocytes results in decreased platelet-leukocyte interactions.

### DETAILED DESCRIPTION OF THE PREFERRED EMBODIMENTS

In an embodiment, the present invention provides compounds for binding sulfatides. The compounds preferably are a polypeptide that contains the N-PTB domain of Disabled-2 (Dab2). Disabled-2 (Dab2) acts as an adaptor protein in multiple pathways, including endocytosis (Maurer et al., Journal of cell science 119, 4235-4246 (2006); and Spudich et al., Nature cell biology 9, 176-183 (2007)) and canonical Wingless Type (Wnt) signaling (Hocevar et al., The EMBO Journal 20:2789-2801 (2001); and Prunier et al., Growth factors (Chur, Switzerland) 22: 141-150 (2004)). Structurally, Dab2 contains two functionally relevant domains: the N-PTB domain and a C-terminal proline-rich domain (PRD) (Yun et al., The Journal of biological chemistry 278:36572-36581 (2003); and Cheong et al., BMC developmental biology 6:3 (2006)). The PRD domain inhibits mitogenic Ras monomeric GTPase pathway activation by binding to the Growth factor Receptor Bound protein-2 (Grb2) (Hocevar et al., The EMBO journal 22:3084-3094 (2003)). The N-PTB domain is a member of the Dab Homology (DH) domain family, which mediates binding to specific peptides and lipids (Yun et al.). The PTB domain mediates Dab2 interaction with Smad2 and Smad3 in the Transforming Growth Factor β (TGFβ) pathway as well as Axin and Dishevelled-3 binding in the canonical Wnt pathway (Maurer et al.). The N-PTB domain specifically binds phosphatidylinositol 4,5-bisphosphate (PtdIns(4,5)P₂) as well as other peptide sequences (Yun et al.; and Homayouni et al., J Neurosci 19:7507-7515 (1999)).

The present inventors have discovered that the N-PTB domain also binds sulfatides regulating platelet aggregation. Sulfatide is a sulfated galactosylceramide synthesized by cerebroside sulfotransferase containing a ceramide backbone with a sulfate head group and two fatty acid tails that vary in length from 18-24 carbons in length (Guchhait et al., Thromb Haemost 99:552-557 (2008)). The ceramide backbone and sulfated head are the same in all sulfatides, while the fatty acid can vary. An example of a sulfatide is shown in Formula I.

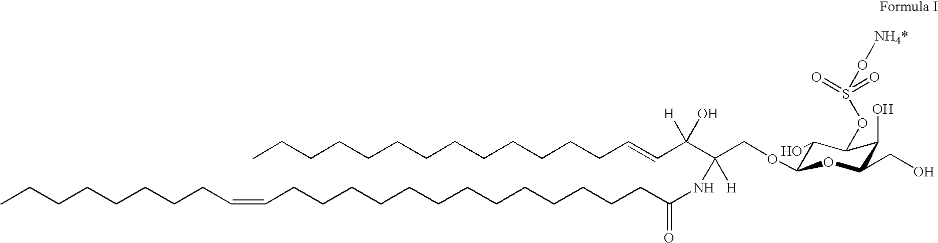

Sulfatide lipids are located predominantly on the outer leaflet of the plasma membrane in glandular epithelial cells, neuronal cells, erythrocytes, platelets, and pancreatic islet cells. Sulfatides have been shown to regulate protein localization as well as cellular adhesion during platelet activation (Guchhait et al.; and Ishizuka et al., Progress in lipid research 36:245-319 (1997)). For instance, sulfatides mediate clustering of voltage-gated ion channels in mouse neuronal cells (Ishibashi et al., J Neurosci 22:6507-6514(2002)). Knockout mice incapable of synthesizing sulfatide lipids exhibit abrogated K+ ion channel localization along axons (Ishibashi et al.). In the absence of sulfatides, the localization of contactin associated protein, an axonal adhesion mediator, is disrupted in neuronal axons. This results in diffuse distribution of the protein and disrupted K+ ion channel clustering (Ishibashi et al.). Thus, sulfatides can function in the localization of cellular proteins. During platelet aggregation, sulfatides bind to P-selectin, an adhesion protein present on activated platelet membranes, to stabilize the bridge between adjacent platelets (Merten et al., Circulation 104:2955-2960 (2001)).

Preferably, the compounds contain at least one of the sulfatide binding domains within the N-PTB domain. A first sulfatide binding domain include amino acids 24-31 of SEQ ID NO: 1. A second sulfatide binding domain include amino acids 49-54 of SEQ ID NO: 1. The compound can also include amino acid substitutions in the sulfatide binding domains without adversely affecting their binding with sulfatides. Preferably, the mutation can include substitution without significantly affecting the binding of the compound with sulfatides. The compound preferably binds sulfatide with a dissociation constant (K_{D}) of at least about of ˜1.93×10⁻⁶ M.

The compounds of the present invention can include multiple sulfatide binding domains separated by linkers. In an example, the compound can include amino acids 24-54 of SEQ ID NO: 1, which includes the first and second sulfatide binding domains separated by a linker. Several of these binding domains ca be linked in series to form a peptide containing at least three, preferably at least four, sulfatide binding motifs. By increasing the number of sulfatide binding motifs in the compound, it is possible to increase the affinity compound for sulfatide, and thus, the effectiveness of the compound in preventing platelet aggregation, preferably secondary platelet aggregation. Linking group libraries for use in activated drug delivery are known to those of skill in the art and may be based on enzyme kinetics, prevalence of active enzyme, and cleavage specificity of the selected disease-specific enzymes (see e.g., technologies of established by VectraMed, Plainsboro, N.J.). Such linkers may be used in modifying the compounds of the present invention for therapeutic delivery. Although it is preferred that the compound contains both the first and second sulfatide binding domains, it can contain one of the two sulfatide binding domains, or a series thereof. In an embodiment, the compounds of the present invention have the following structure:

[D₁-L₁-D₂]ₙ

where D₁ and D₂ can be the same or different and denote sulfatide binding domains, L₁ denotes a linker, and n is an integer greater than 1, preferably 4-8, and most preferably 4. Preferred D₁ is SKKEKKKG (amino acids 24-31 of SEQ ID NO: 1); referred D₂ is KYKAKL (amino acids 49-54 of SEQ. ID NO: 1); and preferred L₁ is PEKTDEYLLARFKGDGV (amino acids 32-48 of SEQ. ID NO: 1) or the same sequence having mutations at the putative thrombin cleavage site (amino acids 44 and 45 of SEQ ID NO: 1). Such mutation can be PEKTDEYLLARFAGDGV (SEQ ID NO: 5) or PEKTDEYLLARFAADGV (SEQ ID NO: 6) or PEKTDEYLLARFKADGV (SEQ ID NO: 7). Although SEQ ID NOS: 5-7 show substitution with Ala, other amino acids are also appropriate. D1 and/or D2 can also be SKKEKKAG (SEQ ID NO: 2) or SKKEKKAA (SEQ ID NO: 3) or SKKEKKKA (SEQ ID NO: 4) where the putative thrombin cleavage site, Lys30 and/or Gly31 of SEQ ID NO: 1, is substituted with Ala. Although SEQ ID NOS: 2-3 show substitution with Ala, other amino acids are also appropriate. In certain embodiments, a linker can also be place between the repeating unit [D₁-L₁-D₂], which can be the same or different from L₁.

The compounds useful in the invention can be linear, or may be circular or cyclized by natural or synthetic means. For example, disulfide bonds between cysteine residues may cyclize a peptide sequence. Bifunctional reagents can be used to provide a linkage between two or more amino acids of a peptide. Other methods for cyclization of peptides, such as those described by Anwer et al. (Int. J Pep. Protein Res. 36:392-399, 1990) and Rivera-Baeza et al. (Neuropeptides 30:327-333, 1996) are also known in the art.

The compounds of the invention, modified with non-peptide moieties that provide a stabilized structure or lessened biodegradation, are also contemplated. Peptide mimetic analogs can be prepared based on the compound of the present invention by replacing one or more amino acid residues of the protein of interest by non-peptide moieties. Preferably, the non-peptide moieties permit the peptide to retain its natural confirmation, or stabilize a preferred, e.g., bioactive confirmation. One example of methods for preparation of non-peptide mimetic analogs from peptides is described in Nachman et al., Regul. Pept. 57:359-370 (1995). It is important that any modification does not affect the sulfatide binding property of the compound. As such, it is preferred that any amino acid modification does not occur within the sulfatide binding domains or motifs of the compound. The term “peptide” as used herein embraces non-peptide analogs, mimetics and modified peptides.

The compounds of the present invention may be modified in order to improve their efficacy. Such modification of the compounds may be used to decrease toxicity, increase bioavailability, or modify biodistribution. A strategy for improving drug viability is the utilization of water-soluble polymers. Various water-soluble polymers have been shown to modify biodistribution, improve the mode of cellular uptake, change the permeability through physiological barriers, and modify the rate of clearance from the body (Greenwald et al., Crit Rev Therap Drug Carrier Syst. 2000; 17:101-161; Kopecek et al., J Controlled Release, 74:147-158, 2001). To achieve either a targeting or sustained-release effect, water-soluble polymers have been synthesized that contain drug moieties as terminal groups, as part of the backbone, or as pendent groups on the polymer chain.

For example, polyethylene glycol (PEG), has been widely used as a drug carrier, given its high degree of biocompatibility and ease of modification (Harris et al., Clin Pharmacokinet. 2001; 40(7):539-51). Attachment to various drugs, proteins, and liposomes has been shown to improve residence time and decrease toxicity (Greenwald et al., Crit Rev Therap Drug Carrier Syst. 2000; 17:101-161; Zalipsky et al., Bioconjug Chem. 1997; 8:111-118). PEG can be coupled to active agents through the hydroxyl groups at the ends of the chain and via other chemical methods; however, PEG itself is limited to at most two active agents per molecule. In a different approach, copolymers of PEG and amino acids were explored as novel biomaterials which would retain the biocompatibility properties of PEG, but which would have the added advantage of numerous attachment points per molecule (providing greater drug loading), and which could be synthetically designed to suit a variety of applications (Nathan et al., Macromolecules. 1992; 25:4476-4484; Nathan et al., Bioconj. Chem. 1993; 4:54-62).

The compounds encompassed by the present invention may also be attached to magnetic beads or particles (preferably nano-particles) to control distribution of the compound. Such compounds can specifically be targeted using a magnetic field, which naturally increases the effectiveness of the compounds. Methods of attaching peptides to magnetic beads are known in the art and are disclosed, for example in U.S. Pat. No. 5,858,534.

The compounds encompassed by the present invention may be produced by conventional automated peptide synthesis methods or by recombinant expression. General principles for designing and making proteins are well known to those of skill in the art.

The peptides encompassed by the present invention can be made in solution or on a solid support in accordance with conventional fmoc-based techniques. The peptides can be prepared from a variety of synthetic or enzymatic schemes, which are well known in the art. Where short peptides are desired, such peptides are prepared using automated peptide synthesis in solution or on a solid support in accordance with conventional techniques. Various automatic synthesizers are commercially available and are used in accordance with known protocols. See, for example, Stewart and Young, Solid Phase Peptide Synthesis, 2d. ed., Pierce Chemical Co., (1984); Tam et al., J. Am. Chem. Soc., 105:6442, (1983); Merrifield, Science, 232: 341-347, (1986); and Barany and Merrifield, The Peptides, Gross and Meienhofer, eds, Academic Press, New York, 1-284, (1979); Fields, (1997) Solid-Phase Peptide Synthesis. Academic Press, San Diego); Andersson et al., Large-scale synthesis of peptides. Biopolymers (Pept. Sci.), 55, 227-250 (2000); Burgess et al., J. Pept. Res., 57, 68-76, (2001); Peptides for the New Millennium, Fields, J. P. Tam & G. Barany (Eds.), Kluwer Academic Publisher, Dordrecht. Numerous other documents teaching solid phase synthesis of peptides are known to those of skill in the art and may be used to synthesis epitope arrays from any allergen.

For example, the peptides are synthesized by solid-phase technology employing an exemplary peptide synthesizer such as a Model 433A from Applied Biosystems Inc. This instrument combines the FMOC chemistry with the HBTU activation to perform solid-phase peptide synthesis. Synthesis starts with the C-terminal amino acid. Amino acids are then added one at a time till the N-terminus is reached. Three steps are repeated each time an amino acid is added. Initially, there is deprotection of the N-terminal amino acid of the peptide bound to the resin. The second step involves activation and addition of the next amino acid and the third step involves deprotection of the new N-terminal amino acid. In between each step there are washing steps. This type of synthesizer is capable of monitoring the deprotection and coupling steps.

At the end of the synthesis the protected peptide and the resin are collected, the peptide is then cleaved from the resin and the side-chain protection groups are removed from the peptide. Both the cleavage and deprotection reactions are typically carried out in the presence of 90% TFA, 5% thioanisole and 2.5% ethanedithiol. After the peptide is separated from the resin, e.g., by filtration through glass wool, the peptide is precipitated in the presence of MTBE (methyl t-butyl ether). Diethyl ether is used in the case of very hydrophobic peptides. The peptide is then washed a plurality of times with MTBE in order to remove the protection groups and to neutralize any leftover acidity. The purity of the peptide is further monitored by mass spectrometry and in some case by amino acid analysis and sequencing.

The peptides also may be modified, and such modifications may be carried out on the synthesizer with very minor interventions. An amide could be added at the C-terminus of the peptide. An acetyl group could be added to the N-terminus. Biotin, stearate and other modifications could also be added to the N-terminus.

The purity of any given peptide, generated through automated peptide synthesis or through recombinant methods, is typically determined using reverse phase HPLC analysis. Chemical authenticity of each peptide is established by any method well known to those of skill in the art. In certain embodiments, the authenticity is established by mass spectrometry. Additionally, the peptides also are quantified using amino acid analysis in which microwave hydrolyses are conducted. In one aspect, such analyses use a microwave oven such as the CEM Corporation's MDS 2000 microwave oven. The peptide (approximately 2 μg protein) is contacted with e.g., 6 N HCl (Pierce Constant Boiling e.g., about 4 ml) with approximately 0.5% (volume to volume) phenol (Mallinckrodt). Prior to the hydrolysis, the samples are alternately evacuated and flushed with N₂. The protein hydrolysis is conducted using a two-stage process. During the first stage, the peptides are subjected to a reaction temperature of about 100° C. and held that temperature for 1 minute. Immediately after this step, the temperature is increased to 150° C. and held at that temperature for about 25 minutes. After cooling, the samples are dried and amino acid from the hydrolysed peptides samples are derivatized using 6-aminoquinolyl-N-hydroxysuccinimidyl carbamate to yield stable ureas that fluoresce at 395 nm (Waters AccQ Tag Chemistry Package). In certain aspects, the samples are analyzed by reverse phase HPLC and quantification is achieved using an enhanced integrator.

In certain embodiments, the peptides of the present invention are made using FMOC solid-phase synthetic methods such as those described above. However, it is also contemplated that those skilled in the art also may employ recombinant techniques for the expression of the proteins wherein a nucleotide sequence which encodes a peptide of the invention is inserted into an expression vector, transformed or transfected into an appropriate host cell and cultivated under conditions suitable for expression as described herein below. Recombinant methods are especially preferred for producing longer polypeptides that comprise peptide sequences of the invention. Recombinant techniques are well known in the art. For example, U.S. Pat. No. 7,659,375 discloses several systems, including prokaryotic, yeast, mammalian and insect cell, for production of recombinant peptides. A preferred recombinant technique for making the N-PTB domain is disclosed in Example 1. Other compounds encompassed by the present invention can similarly be made. As such, in an embodiment, nucleic acid sequences encoding the peptides or polypeptides of the present invention are also contemplated.

The compounds of the present invention have anti-platelet activity, particularly in inhibiting platelet homotypic and/or heterotypic aggregation. As used herein, the term “inhibits platelet aggregation” or the like includes its generally accepted meaning which includes prohibiting, slowing, or reducing the severity or degree of platelet aggregation. Therefore, those compounds are useful for the treatment of disorders that are presently treated using anticoagulant therapy, such as by the use of heparin-based medicaments or other anti-platelet agents such as e.g., Aggrastat™, Aggrenox™, Agrylin™, Flolan™, Integrilin™, Presantine™., Plavix™., Pletal™, REoPro™, Coumdin, Fragmin™, Hep-Lock™, Lovenox™, Miradon™ and the like. Such disorders include pulmonary embolism, unstable angina, myocardial infarction, deep vein thrombosis, atrial fibrillation with embolization, acute and chronic coagulopathies (disseminated intravascular coagulation), for prevention of clotting in arterial and cardiac surgery, for prophylaxis and treatment of peripheral arterial embolism. The compounds encompassed by the present invention are also useful as anti-platelet agents in blood transfusions, extracorporeal circulation, dialysis procedures as well as blood sampling for laboratory procedures. The compounds are also useful to maintain the patency of an indwelling venipucture device that is being used for intermittent injection or infusion therapy or blood sampling. The compounds are also useful in surgical procedures to prevent the formation of blood clots. Such indications are particularly desirable for patients undergoing abdominal surgery to reduce the risk of thromboemolic complications, patients undergoing knee or hip replacement therapy during and following the replacement procedure, as well as a general prophylactic to prevent clot formation at a later stage. The compounds are also useful in the treatment of subjects that are under risk of thromboembolic complications due to severely restricted mobility e.g., during acute illness. Therapeutic methods include both medical therapeutic and/or prophylactic administration, as appropriate.

Of course, it should be understood that the compounds of the present invention may form part of a therapeutic regimen in which the compound is used in combination with a plurality of other therapies for the given disorder. As such, combination therapy is specifically contemplated. In combination therapy, the compound is administered with another anticoagulant or anti-platelet agent which includes, but is not limited to Aggrastat™, Aggrenox™, Agrylin™, Flolan™, Integrilin™, Presantine™., Plavix™., Pletal™, REoPro™, Coumdin, Fragmin™, Hep-Lock™, Lovenox™, Miradon™, tinzaparin, certoparin, parnaparin, nadroparin, ardeparin, enoxaparin, reviparin, dalteparin, and fraxiparin.

From the above discussion, it should be understood that the disorder that may be treated by the compositions of the present invention are limited only by the fact that the disorder needs a therapeutic intervention, which inhibits platelet aggregation. The doses of the agent may be modified for each individual subject. For particular guidance on the routes of administration, and uses those of skill in the art are referred to the Physician's Desk Reference for generalized descriptions of formulations, routes of administration and patient monitoring used for the agents.

Accordingly, in an embodiment of the present invention, the compound can be used to inhibit platelet aggregation. In this method, the compound is contacted with platelet, preferably activated platelet. The compound, thus, binds sulfatides and inhibits platelet aggregation by 1) binding with surface α_{IIb}β₃ integrin to limited platelet-platelet binding (homotypic aggregation); and 2) binding to sulfatides to inhibit platelet-leukocyte binding (heterotypic aggregation).

In another embodiment, the compound of the present invention can be used to inhibit platelet-leukocyte binding. In this method, the compound is contacted with platelets, preferably activated platelet. Here, the compound binds sulfatides to inhibit heterotypic aggregation.

Specific amounts and route of administration may vary, and will be determined in the clinical trial of these agents. However, it is contemplated that those skilled in the art may administer the compounds of the present invention directly, such as by the intravenous route, to effect contact of the platelet, preferably activated platelet, with the compounds to achieve anti-platelet effect.

Pharmaceutical compositions for administration according to the present invention can comprise the compound of the present invention alone or in combination with other anticoagulants or antiplatelet agents. Regardless of whether the active component of the pharmaceutical composition is a compound alone or in combination with another active agent, each of these preparations is in some aspects provided in a pharmaceutically acceptable form optionally combined with a pharmaceutically acceptable carrier. These compositions are administered by any methods that achieve their intended purposes. Individualized amounts and regimens for the administration of the compositions for the treatment of the given disorder are determined readily by those with ordinary skill in the art using assays that are used for the diagnosis of the disorder and determining the level of effect a given therapeutic intervention produces.

It is understood that the suitable dose of a composition according to the present invention will depend upon the age, health and weight of the recipient, kind of concurrent treatment, if any, frequency of treatment, and the nature of the effect desired. However, the dosage is tailored to the individual subject, as is understood and determinable by one of skill in the art, without undue experimentation. This typically involves adjustment of a standard dose, e.g., reduction of the dose if the patient has a low body weight.

The total dose of therapeutic agent may be administered in multiple doses or in a single dose. In certain embodiments, the compositions are administered alone, in other embodiments the compositions are administered in conjunction with other therapeutics directed to the disease or directed to other symptoms thereof.

In some aspects, the pharmaceutical compositions of the invention are formulated into suitable pharmaceutical compositions, i.e., in a form appropriate for applications in the therapeutic intervention of a given disease. Methods of formulating proteins and peptides for therapeutic administration also are known to those of skill in the art. Administration of these compositions according to the present invention will be via any common route so long as the target tissue is available via that route. Most commonly, these compositions are formulated for oral administration. However, other conventional routes of administration, e.g., by subcutaneous, intravenous, intradermal, intramusclar, intramammary, intraperitoneal, intrathecal, intraocular, retrobulbar, intrapulmonary (e.g., term release), aerosol, sublingual, nasal, anal, vaginal, or transdermal delivery, or by surgical implantation at a particular site also is used particularly when oral administration is problematic. The treatment may consist of a single dose or a plurality of doses over a period of time.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. In some aspects, the carrier is a solvent or dispersion medium containing, for example, water, ethanol, polyol (for example, glycerol, propylene glycol, and liquid polyethylene glycol, and the like), suitable mixtures thereof, and vegetable oils. The proper fluidity is maintained, for example, by the use of a coating, such as lecithin, by the maintenance of the required particle size in the case of dispersion and by the use of surfactants. The prevention of the action of microorganisms is brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions is brought about by the use in the compositions of agents delaying absorption, for example, aluminum monostearate and gelatin.

Sterile injectable solutions are prepared by incorporating the compounds of the present invention in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution is suitably buffered if necessary and the liquid diluent first rendered isotonic with sufficient saline or glucose. These particular aqueous solutions are especially suitable for intravenous, intramuscular, subcutaneous and intraperitoneal administration. Those of ordinary skill in the art will readily optimize effective dosages and administration regimens as determined by good medical practice and the clinical condition of the individual patient.

The frequency of dosing will depend on the pharmacokinetic parameters of the compounds and the routes of administration. The optimal pharmaceutical formulation will be determined by one of skill in the art depending on the route of administration and the desired dosage. Such formulations may influence the physical state, stability, rate of in vivo release and rate of in vivo clearance of the administered agents. Depending on the route of administration, a suitable dose is calculated according to body weight, body surface areas or organ size. The availability of animal models is particularly useful in facilitating a determination of appropriate dosages of a given therapeutic. Further refinement of the calculations necessary to determine the appropriate treatment dose is routinely made by those of ordinary skill in the art without undue experimentation, especially in light of the dosage information and assays disclosed herein as well as the pharmacokinetic data observed in animals or human clinical trials.

Typically, appropriate dosages are ascertained through the use of established assays for determining blood levels in conjunction with relevant dose response data. The final dosage regimen will be determined by the attending physician, considering factors which modify the action of drugs, e.g., the drug's specific activity, severity of the damage and the responsiveness of the patient, the age, condition, body weight, sex and diet of the patient, the severity of any infection, time of administration and other clinical factors. As studies are conducted, further information will emerge regarding appropriate dosage levels and duration of treatment for specific diseases and conditions. Those studies, however, are routine and within the level of skilled persons in the art.

It will be appreciated that the pharmaceutical compositions and treatment methods of the invention are useful in fields of human medicine and veterinary medicine. Thus, the subject to be treated is a mammal, such as a human or other mammalian animal. For veterinary purposes, subjects include for example, farm animals including cows, sheep, pigs, horses and goats, companion animals such as dogs and cats, exotic and/or zoo animals, laboratory animals including mice rats, rabbits, guinea pigs and hamsters; and poultry such as chickens, turkeys, ducks and geese.

In yet another embodiment, the present invention relates to a method for promoting platelet aggregation. In essence, this method is the inverse of the method for inhibiting platelet aggregation. This method involves contacting sulfatides with platelets, preferably activated platelet to promote platelet aggregation. As used herein, the term “promotes platelet aggregation” or the like includes its generally accepted meaning which includes permitting, speeding, or increasing the severity or degree of platelet aggregation. The sulfatides may be administered and formed into pharmaceuticals as mentioned above for the compounds of the invention.

Without further description, it is believed that one of ordinary skill in the art can, using the preceding description and the following illustrative examples, make and utilize the compounds of the present invention and practice the claimed methods. The following examples are given to illustrate the present invention. It should be understood that the invention is not to be limited to the specific conditions or details described in those examples.

### Example 1

### Materials and Methods

### Chemicals

The following is a list of chemicals used and their suppliers: brain sulfatides, dipalmitoyl phosphatidylcholine (PC), dipalmitoyl phosphatidylethanolamine (PE), dipalmitoyl phophatidyserine (PS) (Avanti Polar Lipids), cholesterol (Sigma), dipalmitoyl and dioctanoyl PtdIns(4,5)P₂ (Cayman Chemicals). All other chemicals were analytical reagent grade.

### DNA Cloning, Plasmids and Protein Expression and Purification

Flag-tagged full-length human Dab2 cDNA construct was cloned into a pCS2+MT vector. The N-PTB domain (residues 1-241) cDNA construct was cloned into a pGEX6P1 vector (GE Healthcare). Site directed mutagenesis of Dab2 and its PTB domain were performed using the Quick-Change exchange protocol (Stratagene). Expression and purification of all GST-fusion proteins from E. coli Rosetta cells (Novagen) on glutathione beads were performed as previously described (Sweede et al., Biochem., 2008 Dec. 23; 47(51):13524-36). Purity of all proteins was over 95% as judged by SDS-PAGE gels.

### Protein-Lipid Overlay Assay

Membrane strips (SphingoStrips™) spotted with 100 pmol of sphingolipids were purchased from Echelon Research Laboratories. Membrane strips were incubated with 0.1 μg/ml of the Dab2 PTB domain or its mutants in 20 mM Tris-HCl (pH 8.0), 150 mM NaCl, 0.1% Tween-20 and 3% fatty acid-free bovine serum albumin overnight at 4° C. Following four washes with the same buffer, proteins bound to the membrane strips were probed with rabbit anti-GST antibody (Santa Cruz Biotech). Donkey anti rabbit-horse radish peroxidase (HRP) antibody was obtained from GE Healthcare. Detection was carried out using ECL reagent (Pierce). Also, lipid strips were prepared by spotting 1 μl of either sulfatides or PtdIns(4,5)P₂ dissolved in chloroform:methanol:water (1:2:0.8 and 65:35:8, respectively) onto Hybond-C extra membranes (GE Healthcare) and protein binding was monitored as described above. Bound protein was quantified using the AlphaImager program and binding was calculated as a percentage of GST-PTB binding to 100 pmoles of spotted lipid.

### Liposome-Binding Assay

Lipid stocks including sulfatides, PE, PS, and PtdIns(4,5)P2 were dissolved in chloroform:methanol:water (1:2;0.8, 65:35:8, 65:35:4 and 65:35:8, respectively), whereas cholesterol and PC were dissolved in chloroform:methanol (1:1). Sulfatides liposomes were prepared in a weight ratio of 1:1:1:4 of PC:PE:cholesterol:sulfatides. PtdIns(4,5)P₂ liposomes were prepared in a percent ratio of 50:20:10:10:10 of PC:PE:PS:cholesterol:PtdIns(4,5)P₂. Controls were prepared by adjusting the ratios with PC. Lipid films were generated by lyophilization overnight and hydrated in 20 mM Tris-HCl (pH 6.8), 100 mM NaCl, 2 mM dithiothreitol (DTT) at 1 mg/mL and freeze-thawed 3 times. Liposomes were sonicated and further pelleted and suspended in 4 mg/mL. Ten μg of protein were incubated with 125 μg of total lipids for 20 min at RT. Liposome-bound and free-protein samples were separated by centrifugation and analyzed by SDS-PAGE. Bands were quantified using AlphaImager, and the percentage of total protein each fraction contained was calculated from the total protein.

### Surface Plasmon Resonance

Surface plasmon resonance (SPR) binding experiments were performed on a BIAcore X instrument using L1 sensorchips coated with ˜0.5 mM of mixed sulfatides or PtdIns(4,5)P₂ size calibrated liposomes. N-PTB domain or its mutants binding experiments were performed in a degassed solution containing 10 mM Tris-HCI (pH 7.4) and 150 mM NaCl. This buffer was used during equilibration, association and dissociation phases. Proteins were added to this buffer at the indicated concentrations. Regeneration of the phospholipid bilayer after dissociation phase was carried out using 20 mM NaOH. For competition SPR, increasing molar amounts of dioctanoyl PtdIns(4,5)P₂ were preincubated with 5 μM N-PTB for 20 min at room temperature. The entire reaction was then exposed to an L1 sensorchip containing immobilized sulfatide liposomes (˜0.5 mM).

### Thrombin Limited Proteolysis

Sulfatides- and PtdIns(4,5)P₂-enriched liposomes were incubated with 10 μg of protein for 30 min at room temperature, the reaction centrifuged, and pellets suspended in 50 μl of liposome binding buffer. Thrombin (0.05 units/μg protein) was added to both supernatants and pellets, and aliquots were taken at 0, 8, 12, 16, and 20 h after digestion. Reactions were stopped by the addition of Laemmli buffer and analyzed by SDS-PAGE.

### Platelet Immunofluorescence Analysis

Washed platelets (50 μl of 3×10⁻⁸ platelets/mL in Tyrode's albumin buffer) were incubated with 1.9 μM of PTB domain constructs for 5 min at room temperature in the presence of 0.25 g/L fibrinogen. Aggregation was initiated by the addition of 10 μM TRAP. Reactions were incubated at room temperature unless otherwise indicated. Platelets were fixed with 0.4 mL of 3.7% formaldehyde in Phosphate Buffered Saline (PBS) for 30 min. Next, 20% goat serum was added to the fixed platelets, and 60% of the total fixed reaction was centrifuged using a Shandon cytospinner onto a Shandon coated cytoslide to achieve the appropriate platelet density for analysis. Slides were then washed twice with PBS for 10 min. Platelets were permeabilized with 0.5% Triton X-100 in PBS for 10 min and then blocked with 20% goat serum in PBS with 0.1% Triton X-100 for 1 h at room temperature. Platelets were washed 3 times with PBS with 0.1% Triton for 10 minutes and further incubated overnight with the anti-Dab2 antibody 1:100 (anti-p96; BD Transduction) in PBS with 0.1% Triton X-100 and 10% goat serum at 4° C. in a humidity chamber. Platelets were then washed and incubated with the anti-mouse Cy-3 conjugated goat antibody 1:250 and Alexa488 conjugated phalloidin (25 μg/mL) in PBS with 0.1% Triton X-100 and 10% goat serum for 2 h. Platelets were washed again and observed on an Olympus IX71 microscope using the Softworx Applied Precision software.

Mean pixel intensity was determined for 400 platelets from four fields each for 3 min and 10 min of treatments by manually drawing circles around individual platelets using the 2D Polygon tool in the Softworx software. For each time point, a threshold of significant mean pixel intensity was defined for the experiment. The percentage of countable platelets to exceed the threshold was calculated for each field and the average of four fields was computed with standard deviation. The ratio of internalization:binding is the quotient of these averages for a treatment.

### Platelet Adhesion Assay

Human washed platelets were incubated with BSA, Dab2 PTB or Dab2 PTB^{4M} (1.9 μM each) for 5 min at room temperature in the presence of 0.25 g/L fibrinogen. Aggregation was initiated by the addition of 10 μM TRAP, and reactions were incubated 20 min at 37° C. Samples were fixed with 3.7% formaldehyde in PBS, stained with Wright stain, and photographed. Wright stain was eluted with 20% ethanol, and absorbance was quantified at 415 nm.

### CD Spectroscopy

Spectra were collected using a Jasco J-720 spectropolarimeter. Far-UV CD spectra were measured for Dab2 PTB constructs (5 μM in 5 mM Tris, 100 mM potassium fluoride, 100 μM DTT, pH 6.8) using a 1 mm-slit-width cuvette. Five accumulated scans were recorded for each sample between 190 and 260 nm at 20° C. in increments of 0.5 nm. Background of buffer alone was subtracted from each spectrum. Raw data were converted to mean residue ellipticity and analyzed for secondary structure composition using DICHROWEB and deconvoluted using CDSSTR.

### Liposome Preparation

Stocks of brain sulfatides, phosphatidylcholine (PC), phosphatidylethanolamine (PE) (Avanti Polar Lipids) and cholesterol (Sigma) were prepared in organic solvents per manufacturer instructions. Liposomes were prepared in the absence and presence of sulfatides as described by Larsen et al. (Cell 1989; 59:305-312).

### Blood Collection and Platelet Purification

Whole blood was collected from healthy volunteers by venipuncture into 10% acid citrate dextrose blood collection tubes. Whole blood was then centrifuged at 200 g for 15 min to separate platelet rich plasma (PRP) from contaminating erythrocytes. PRP was removed and centrifuged at 2,200g for 10 min to remove platelets from the plasma. Platelet poor plasma was removed and platelets suspended in Tyrode's albumin buffer (10 mM HEPES (pH 7.4), 134 mM NaCl, 12 mM NaHCO₃, 2.9 mM KCl, 0.34 mM Na₂HPO₄ and 1 mM MgCl₂,) containing 10 U/mL heparin and 0.5 μM prostaglandin (PGI2). Platelets were washed again in Tyrode's albumin buffer containing PGI2 and counted.

### Device Design

The microfluidic device consisted in a simple straight channel 500 μm wide, 50 μm deep and 3.62 cm long. A silicon master stamp was fabricated on a <100> silicon substrate following a previously described process (Ross et al. Ann. Rev. Med. 1987, 38:71-79). Flow was driven using a micro-syringe pump (Cole-Parmer) with a 1 mL syringe connected to the channel inlet by 30 cm of Cole-Parmer gauge 20 Teflon tubing. After priming the system with the sample, the pump was set to 0.05 mL/h (equivalent to an average velocity of 0.55 mm/sec in the channel), which is in the range of in vivo blood velocity (0.1-1.5 mm/sec (9)) and causes a shear rate of 70 sec−1. This flow rate was maintained for 1 min prior to the experiments. Platelets flowing through the channel were monitored using an inverted light microscope (DMI 6000B, Leica Microsystems) equipped with a digital camera (DFC420, Leica Microsystems).

### Flow Cytometry

Washed platelets (2.5×105 platelets/μL) were kept unactivated or either activated with ADP (30 μM) or TRAP (10 μM). Both unactivated and activated platelets were incubated for 10 min at 23° C. with liposomes (50 μg/mL) either without (liposomes control) or with sulfatides, and N-PTB constructs (1 μM). Reactions were fixed with 1% formalin and incubated with PE- labeled CD62p anti-P-selectin (BioLegend) or FITC-labeled PAC-1 anti-integrin receptor (BD Transduction) antibodies. Bound antibodies were quantified using a FacsAria flow cytometer.

### Leukocyte Purification and Platelet-Leukocyte Aggregation Assay

Whole blood was collected as indicated above and centrifuged at 200 g to separate the blood from plasma and red blood cell fractions. Plasma and buffy coat layer were carefully removed and diluted 1:1 with PBS. The dilution was then layered onto a Ficoll Plus gradient and spun at 200 g for 20 min. The enriched platelet and leukocyte layer was removed, diluted with PBS, centrifuged and the pellet resuspended in Tyrode's Albumin buffer to a concentration of ˜3×105 platelets/μL and 3×103 leukocytes/μL. Isolated platelet-leukocyte mixtures were remained unactivated or activated with ADP (30 μM). Both unactivated and activated platelets were incubated for 10 min at 23° C. with liposomes (50 μg/mL) either without (liposomes control) or with sulfatides, and N-PTB constructs (1 μM). Reactions were fixed with 1% formalin and incubated with APC-labeled CD42b (Biolegend) and FITC-labeled CD45 (Biolegend) antibodies. Bound antibodies were quantified using a FacsAria flow cytometer.

### Example 2

### Conserved Basic Motifs Mediate N-PTB Interaction with Sulfatides

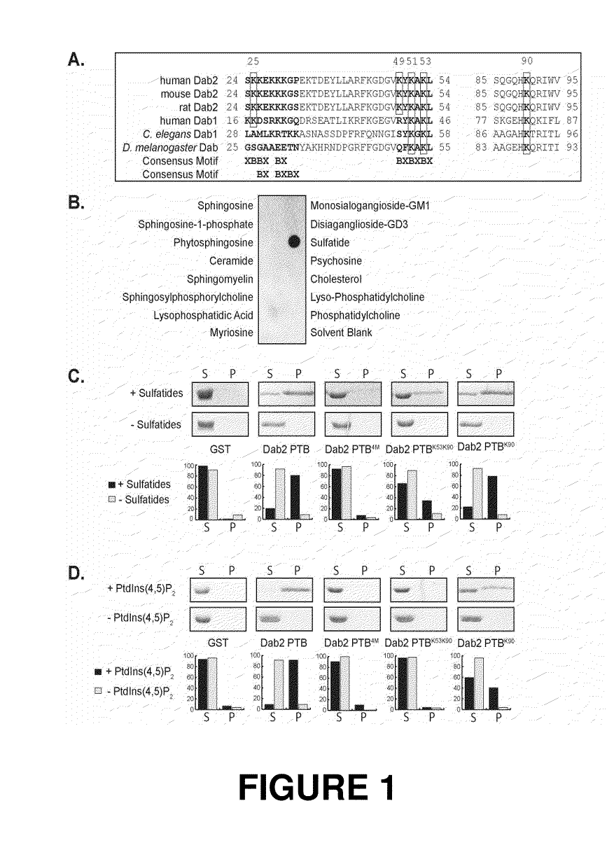

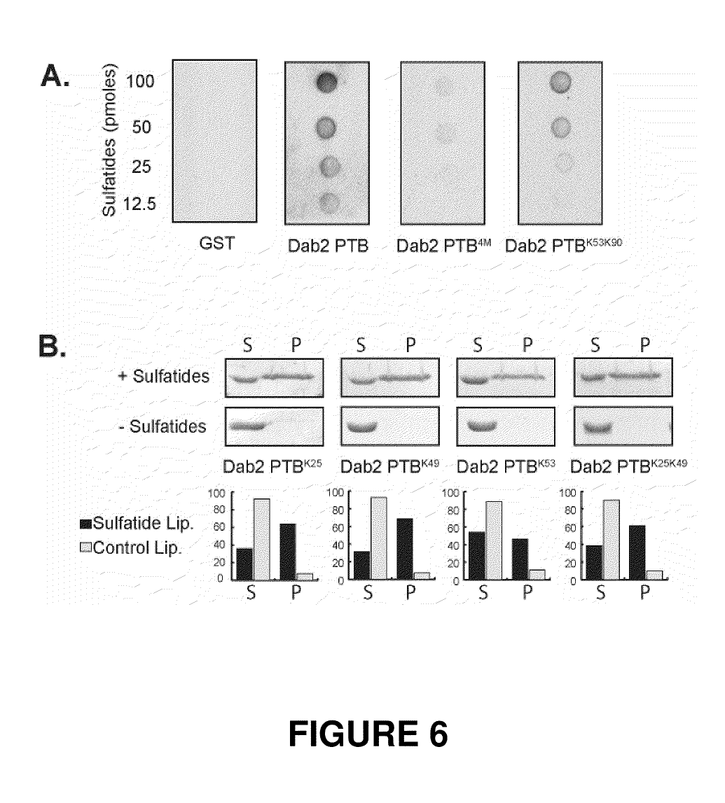

Analysis of the PTB domain amino acid sequence showed the presence of conserved positively charged residues that resembled two characteristic sulfatide-binding sites represented by the XBBXBX and BXBXBX motifs, where B and X indicate basic and any residue, respectively (FIG. 1A) (Sandhoff et al., Biochimica et biophysica acta 1687:52-63 (2005)). Several homeostatic proteins including laminin, VWF, P-selectin and thrombospondin have multidomain regions that interact with sulfatides. For example, Laminin-1 binds to sulfatides through a region that comprises two XBBXBX and three BXBXBX sequences (Taraboletti et al., The Journal of biological chemistry 265:12253-12258 (1990)). Likewise, a patch of positively charged residues in VWF is responsible for sulfatides ligation (Nakayama et al., Int J Hematol. 2008 May; 87(4):363-70.). To test our hypothesis, we analyzed sulfatide binding by the PTB domain in comparison with other sphingolipids commonly found at the plasma membrane. Among sphingolipids, the N-PTB domain preferentially bound to sulfatides (FIG. 1B). As the N-PTB domain targets biological membranes, we employed liposomes enriched with sulfatides to further investigate the interaction. The N-PTB domain bound to sulfatide-enriched liposomes (FIG. 1C) as well as to sulfatides immobilized on membrane strips (FIG. 6A). Single amino acid mutations to neutral Ala designed in the putative sulfatide-binding site (Lys25, Lys49, or Lys53) or combination of two mutations (Lys25 and Lys49) reduced but did not eliminate sulfatide binding (FIG. 6B). However, a substitution of four positively charged residues (Lys25, Lys49, Lys51 and Lys53; Dab2 PTB^{4M}) in the putative sulfatide-binding sites (FIG. 1A) with Ala led to complete abolishment of lipid binding (FIG. 1C). The circular dichroism (CD) spectrum of Dab2 PTB^{4M} (as well as single and double mutants of the same protein) did not exhibit significant changes in the secondary structure content as compared with the wild-type PTB, as shown by the consistency of the percentage of α-helical content (Helix1 and Helix2) and β-strand content (Strand1 and Strand2) (Table 1).

TABLE 1
Secondary structure composition of Dab2 PTB. Predictions were
generated using DICHROWEB and deconvoluted using CDSSTR.

| Construct | Helix 1 | Helix 2 | Strand 1 | Strand 2 | Turns | Unordered | Total | NRMSD |
|---|---|---|---|---|---|---|---|---|
| WT | 8% | 10% | 17% | 10% | 23% | 31% | 99% | 1.5% |
| 4M | 7% | 9% | 20% | 11% | 22% | 31% | 100% | 2.7% |
| K25 | 7% | 10% | 17% | 11% | 24% | 32% | 101% | 2.2% |
| K49 | 8% | 11% | 18% | 10% | 25% | 28% | 100% | 4.4% |
| K53 | 6% | 10% | 17% | 11% | 26% | 30% | 100% | 5.2% |
| K90 | 6% | 9% | 18% | 11% | 24% | 32% | 100% | 2.1% |
| K25K49 | 6% | 10% | 17% | 11% | 24% | 32% | 100% | 1.8% |
| K53K90 | 7% | 9% | 16% | 10% | 26% | 32% | 100% | 4.0% |
| D66E | 7% | 9% | 17% | 10% | 24% | 32% | 99% | 1.8% |

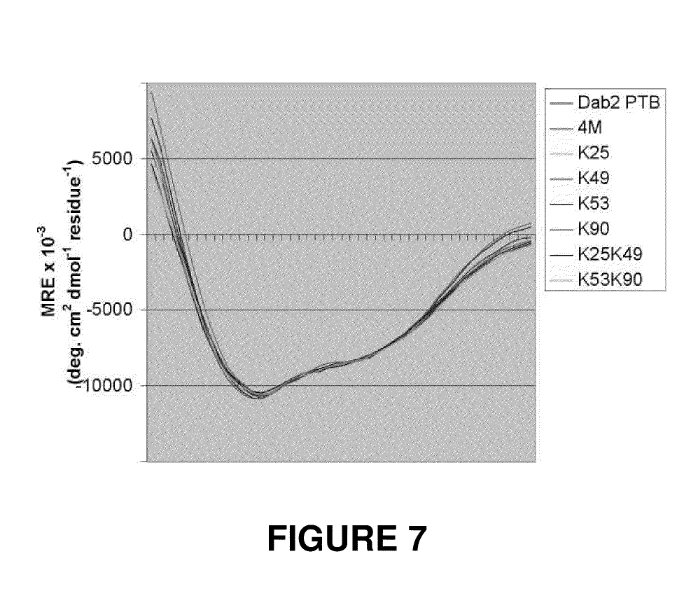

These data indicate that mutations do not significantly perturb the global fold of the protein (FIG. 7 and Table 1). Thus, we conclude that the two positively charged motifs in N-PTB domain play a critical role in sulfatide recognition.

### Example 3

### N-PTB Binds Sulfatides and PtdIns(4,5)P₂ with Comparable Affinities

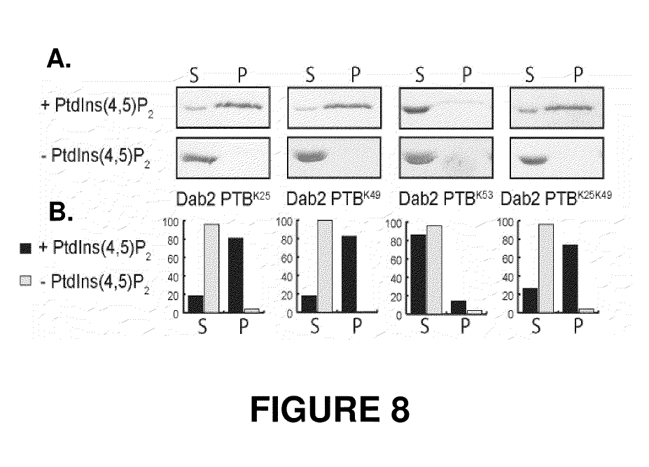

The N-PTB domain is known to bind phosphoinositides with preference to PtdIns(4,5)P₂, independent of the protein interaction site (Howell et al., Molecular and cellular biology 19, 5179-5188 (1999)). Two basic residues (Lys53 and Lys90) have been reported to play a critical role in PtdIns(4,5)P₂ recognition (Yun et al.). Therefore, we examined whether these residues could be also critical for sulfatide binding. Mutation in the PTB domain at both Lys53 and Lys90 to Ala (Dab2 PTB^{K53K90}) reduced sulfatide binding in about 3-fold (FIG. 1C) similarly as observed with a mutation at Lys53 (FIG. 6B), but a single mutation at Lys90 (Dab2 PTB^{K90}) did not (FIG. 1C). As expected, the N-PTB domain bound to PtdIns(4,5)P₂-containing liposomes, whereas PTB^{K53K90} and PTB^{4M} did not (FIG. 1D). Consistent with these results, lipid-protein overlay assays showed that the sulfatide-binding mutant, Dab2 PTB^{4M}, bound very weakly to bind PtdIns(4,5)P₂, whereas PTB^{K53K90} did not bind at all (FIG. 8A). Conversely, with the exception of the Lys53 mutant, single or double mutations in the sulfatide-binding sites in the PTB domain did not significantly affect PtdIns(4,5)P₂ binding (FIG. 8B). Mutations in these residues did not alter the secondary structure of the protein, indicating that they specifically abolished lipid binding (FIG. 7 and Table 1). Overall, these results indicate that Lys53 μlays a critical role in both sulfatide and PtdIns(4,5)P₂ ligation.

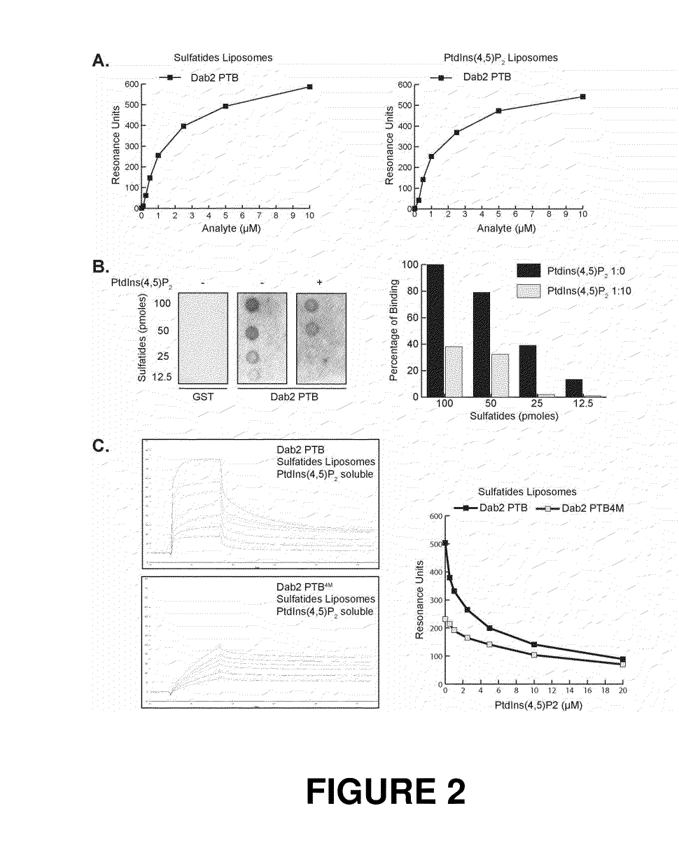

The kinetics of the interaction between the N-PTB domain and the two lipids were investigated by surface plasmon resonance (SPR). The N-PTB domain exhibited sulfatide binding with an estimated dissociation constant (K_{D}) of ˜1.93×10⁻⁶ M (FIG. 2A, left panel). This affinity is close to that reported for sulfatide binding by the Escherichia coli heat-stable enterotoxin b (Beausoleil et al., Receptors & channels 7:401-411 (2001)). Interestingly, the kinetics displayed a reversible binding mode to either immobilized sulfatide or PtdIns(4,5)P₂ liposomes, with Dab2 PTB^{4M} displaying retarded association and dissociation compared to wild type N-PTB (data not shown). The wild type N-PTB kinetic data for both sulfatide and PtdIns(4,5)P₂ fit a two-state binding model with conformational change, but did not fit a 1:1 Langmuir binding model, suggesting a more complex interaction of the PTB domain with its lipid ligands. Likewise, the PTB domain bound PtdIns(4,5)P₂ with a calculated K_{D} of ˜1.5×10⁶M (FIG. 2A, right panel). Dab2 PTB^{4M} exhibited a drastically reduced binding to PtdIns(4,5)P₂ liposomes (data not shown), confirming that sulfatide and PtdIns(4,5)P₂ binding sites overlap in the PTB domain.

### Example 4

### PtdIns(4,5)P₂ Competes with Sulfatides for N-PTB Binding

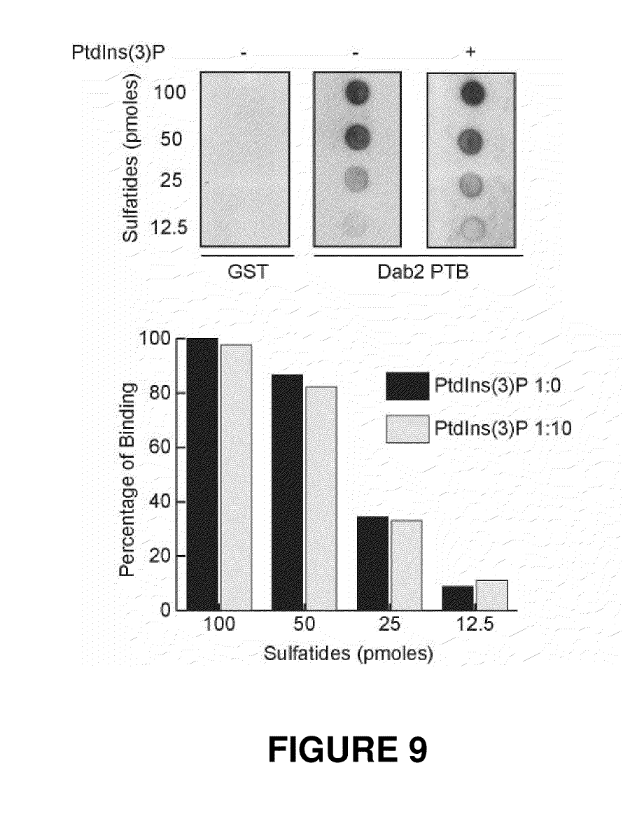

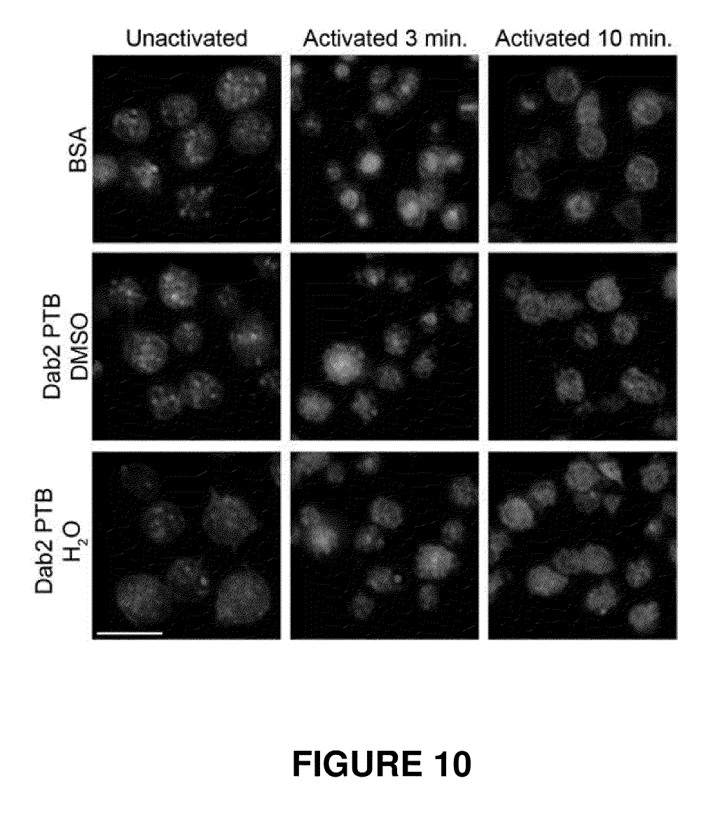

We next asked whether sulfatides and PtdIns(4,5)P₂ compete with each other for binding to the N-PTB domain using a protein-lipid overlay competition assay. Pre-incubation of the N-PTB domain with 10-fold excess of PtdIns(4,5)P₂ reduced sulfatide binding in at least 60% (FIG. 2B), whereas a related phosphoinositide, phosphatidylinositol 3-phosphate (PtdIns(3)P) did not compete with sulfatide for PTB binding (FIG. 9). This observation was further confirmed by competing sulfatides and PtdIns(4,5)P2 for N-PTB domain binding using SPR. Pre-incubation of the protein with PtdIns(4,5)P₂ reduced the affinity for sulfatides with increasing molar excess of PtdIns(4,5)P₂, showing an IC₅₀ of ˜2 μM for N-PTB (FIG. 2C). However, the IC₅₀ was reduced approximately four-fold for Dab2 PTB^{4M} (FIG. 2C).

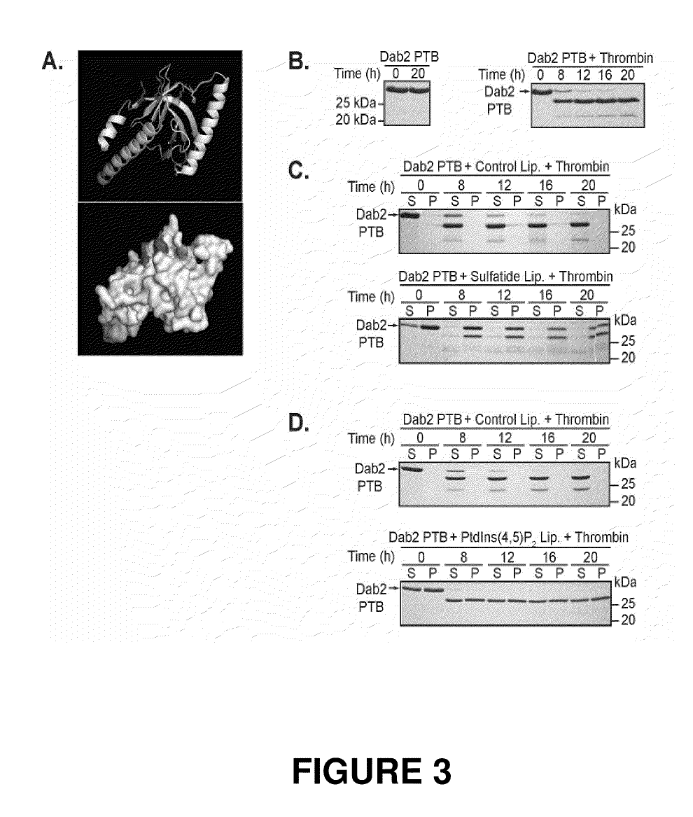

The tertiary structure of the N-PTB domain exhibits a continuous patch of positively charged residues formed by Lys49, Lys51, Lys53 and Lys90 (FIG. 3A) (Lys25 is not present in the reported N-PTB domain crystal structure (Yun et al.)). Two experimental observations can explain the competition of the two lipids for N-PTB domain binding. First, Lys53 is necessary for both sulfatides and PtdIns(4,5)P₂ ligation (FIG. 3A). Second, despite the fact that Lys90 is far from the sulfatide binding motifs, the orientation of its side chain towards the sulfatide-binding site in the tertiary structure of the protein (FIG. 3A) may impair simultaneous binding of both lipids to the protein.

### Example 5

### Sulfatide Binding Protects Dab2 from Thrombin Proteolysis

During platelet activation, Dab2 is released from α-granules and binds to the extracellular region of αIIβ3 integrin on the platelet surface, where it exerts its anti-platelet aggregation activity (Huang et al. 2006). Dab2 is ineffective in inhibiting thrombin-mediated platelet aggregation due to the presence of two thrombin cleavage sites within its PTB domain (Huang et al., Journal of cell science 119:4420-4430 (2006)). In agreement with previous observations of Huang et al., thrombin cleaved the N-PTB domain leaving a ˜25 kW protease-resistant product (FIG. 3B). To understand the role of sulfatides binding by Dab2 during thrombin-mediated platelet aggregation, the N-PTB domain was pre-incubated with liposomes containing sulfatides and the protein-lipid complexes treated with thrombin. Interestingly, sulfatide-enriched liposomes protected bound N-PTB from thrombin cleavage (FIG. 3C). These results suggest that the thrombin cleavage sites in the N-PTB domain are less exposed in the protein when bound to sulfatides and thus binding may stabilize Dab2 during thrombin-mediated platelet activation. In contrast, PtdIns(4,5)P₂ did not protect the PTB domain from thrombin cleavage (FIG. 3D), indicating that the phosphoinositide does not have a direct role on Dab2 stabilization during thrombin-mediated platelet aggregation. Indeed, this result is consistent with the platelet subcellular localization of the lipids. Whereas sulfatides are located at the platelet surface (Merten et al., Circulation 104:2955-2960 (2001), and therefore can protect Dab2 from thrombin cleavage, PtdIns(4,5)P2 is usually found at the cytosolic side of the membrane (Maldonado-Baez et al., Trends Cell Biol 16:505-513 (2006)).

### Example 6

### Sulfatides Compete with the αIIbβ3 Integrin for PTB Binding on the Surface of Activated Platelets

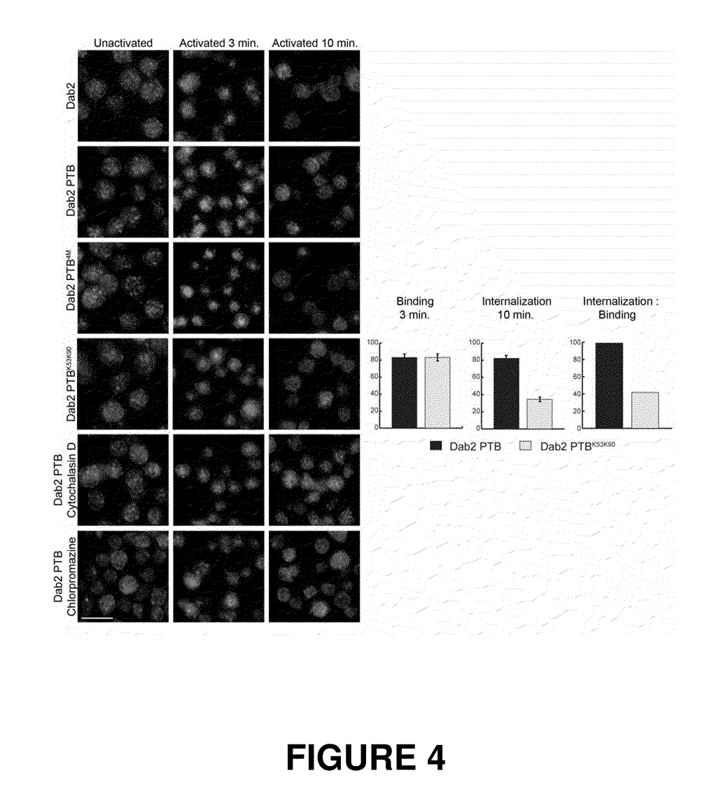

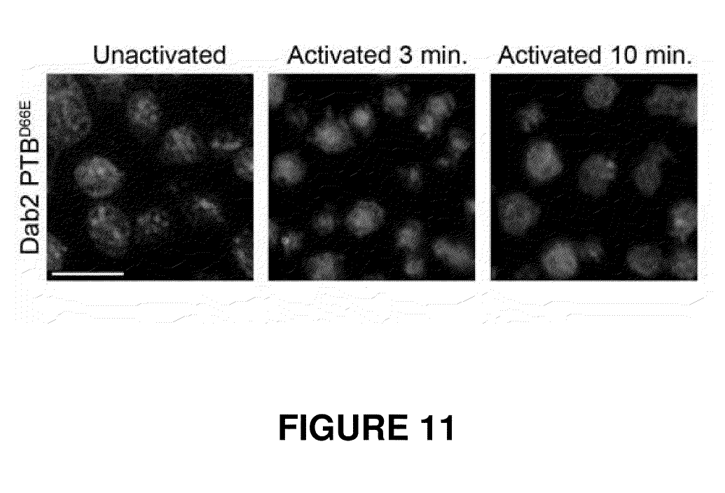

The N-PTB domain has been recently shown to bind to platelet integrin receptors (Huang et al., Journal of cell science 119:4420-4430 (2006)) and to sulfatides (this work); thus, it is likely that two pools of Dab2 can be found at the platelet surface. To investigate this hypothesis, we analyzed the subcellular localization of endogenous Dab2 N-PTB and PTB^{4M} in platelets activated after thrombin receptor-activating peptide (TRAP) stimulation. As expected, the endogenous Dab2 was localized peripherally after 3 min of platelet activation similarly as reported in Huang et al. (2006) (FIG. 4, center panel). Whereas the isolated N-PTB domain was clearly localized at the platelet surface after 3 min followed of TRAP stimulation (FIG. 4, center panel), mutations in the sulfatide-binding site reduced, but did not completely abolish, the localization of the protein at the platelet surface (FIG. 4, center panel). These results suggest that mutations in the sulfatide-binding site abrogate sulfatide binding at the platelet surface. In addition, we propose that the mutant protein can instead bind to the integrin receptor, since the N-PTB domain still presents the RGD (amino acids 64-66) motif, which has been shown to be responsible for binding of the N-PTB domain to the αIIb domain of the integrin receptor (Huang et al. 2006). Previous studies have shown that a Glu mutation at the residue Asp66 in the RGD site at the N-PTB domain abolishes the interaction of Dab2 with the integrin receptor (Huang et al. 2006), without affecting lipid binding (Yun et al.). In our hands, however, PTB^{D66E} showed little decrease in binding to the platelet surface (FIG. 11, center panel).

### Example 7

### Binding to PtdIns(4,5)P₂ Mediates Clathrin-Dependent Endocytosis of Dab2

Interestingly, when platelets were fixed 10 min after their activation, the endogenous Dab2, its N-PTB domain and the PTB^{4M}, to a lesser extent, were internalized (FIG. 4, right panels). Platelets undergo endocytosis through two different mechanisms (Behnke, J Submicrosc Cytol Pathol 24:169-178 (1992)). One mechanism is mediated by clathrin-coated vesicles budding from specialized regions of the platelet plasma membrane called the open canalicular system, which delivers their content to α-granules. For example, the internalization of the aIIbβ3 integrin receptor has been shown to be dependent on its activation, which may be necessary to down-regulate the adhesiveness of activated platelets (Wencel-Drake et al., 87:602-612 (1996)). The second mechanism involves a clathrin-independent degradative pathway (Behnke). Thus, it is possible that after signaling at the platelet surface, the N-PTB domain is recycled by internalization and stored in the α-granules. Interestingly, mutation in the PtdIns(4,5)P₂ binding site in the N-PTB domain, PTBK53K90 , did not significantly affect the membrane localization of the protein (FIG. 4, center panel), indicating that the phosphoinositide is dispensable for the localization of Dab2 at the platelet surface. However, after 10 min post-activation, platelets exhibited a 40% reduced internalization of PTB^{K53K90} (FIG. 4, right panels). This result indicates that the inability of the N-PTB domain to bind PtdIns(4,5)P₂ affects its internalization, consistent with the proposed model in which clustering of adaptor proteins, mediated by the phosphoinositide, facilitate local destabilization and membrane deformation during endocytosis.

Dab2 is implicated in receptor turnover and endocytosis. This function is mediated by its N-PTB domain, which interacts with receptors by their NPXY (where X is any amino acid) motifs (Morris et al., Traffic 2:111-123 (2001)). The N-PTB domain depends upon the presence of phosphoinositides to initiate the clathrin-coated vesicle formation (Mishra et al., The EMBO journal 21:4915-4926 (2002)). To further investigate the mechanism by which the N-PTB domain itself is internalized, we have used chlorpromazine, an inhibitor that specifically blocks clathrin-mediated endocytosis (Rejman et al., Mol Ther 12:468-474 (2005)). Platelets exhibited peripheral N-PTB domain after 3 min of their activation in the presence of the inhibitor (FIG. 4). However, internalization of the protein was inhibited in the presence of chlorpromazine, displaying a similar phenotype to the PTB^{K53K90} mutant (FIG. 4). This observation was confirmed by incubating activated platelets with cytochalasin D, an actin polymerization inhibitor, which completely blocked N-PTB domain internalization (FIG. 4). All together, these results suggest that clathrin-coated vesicles mediate N-PTB domain internalization in platelets.

### Example 8

### Two Pools of Dab2 Exist at the Surface of the Activated Platelet

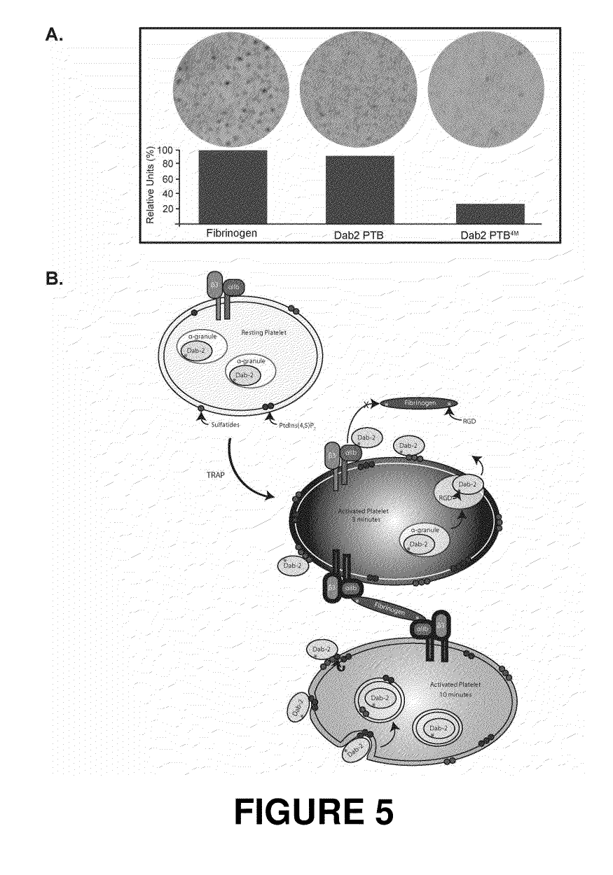

Binding of fibrinogen to platelets triggers platelet aggregation during blood clotting (Jackson, Blood 109:5087-5095 (2007)). It has been recently shown that the N-PTB domain competes with fibrinogen for integrin binding at the platelet surface (Huang et al. 2006). A platelet adhesion assay was carried out using washed platelets on a plate surface in the presence of fibrinogen. To understand whether sulfatide-binding by the N-PTB domain is critical for integrin receptor function, we performed a competition platelet adhesion assay. The N-PTB domain did not significantly affect platelet adhesion to fibrinogen, whereas PTB^{4M} reduced platelet adhesion by 80% (FIG. 5A). These results demonstrate that the sulfatide-binding mutant inhibited platelet adhesion, suggesting that abolition of sulfatide binding makes a pool of N-PTB domain available to compete with fibrinogen for binding to the integrin receptor.

On the basis of our results and previously reported findings, we propose a model to describe the role of both sulfatides and PtdIns(4,5)P₂ in Dab2 function (FIG. 5B). In this model, two pools of Dab2 are found at the platelet surface after its activation. One pool negatively controls platelet aggregation by competing with fibrinogen for binding to the αIIβ3 integrin receptor. This equilibrium depends upon thrombin activation, which cleaves Dab2 at the N-PTB domain favoring platelet aggregation. A second pool of Dab2 is bound to sulfatides and inaccessible for thrombin cleavage. Both pools are internalized upon platelet activation by a clathrin-mediated endocytosis in a PtdIns(4,5)P2-dependent fashion.

Platelets express sulfatides on their surface, which increase after activation (Merten et al.). However, the role of sulfatides during this event is still not clear (Kyogashima, M. Archives of biochemistry and biophysics 426:157-162 (2004)). Whereas sulfatides activate platelets through P-selectin and enhance platelet aggregation (Merten et al., Arteriosclerosis, thrombosis, and vascular biology 25:258-263 (2005)), exogenous sulfatides inhibit platelet function. (Devaiah et al., Blood CoagulFibrinolysis 11:543-550 (2000)). Our finding that sulfatides sequester the N-PTB domain, and therefore Dab2, away from the integrin receptor further defines the role of the lipid in platelet aggregation. In addition, our experiments indicate that internalization of the N-PTB domain can occur not only through αIIbβ3 integrin receptors, but also can be facilitated by sulfatides. This suggests an additional role of sulfatides in recycling Dab2.

Upon platelet activation, activated integrin receptors mediate localized clustering of PtdIns(4,5)P2 in lipid rafts on the inner leaflet of the plasma membrane (Bodin et al., Journal of cell science 118:759-769 (2005)). Furthermore, the “flip-flop” of negatively charged phospholipids from the inner leaflet to the outer leaflet of the plasma membrane increases during the course of platelet activation (McNicol et al., J Pharmacol Exp Ther 281:861-867(1997)). Activated platelets recycle integrin receptor continuously, which serves as a mechanism to down-regulate the adhesiveness of platelets later in aggregation (Wencel-Drake et al.). Our findings suggest that Dab2 is internalized by clathrin-dependent endocytosis, and that this internalization is PtdIns(4,5)P₂-dependent. Furthermore, our experiments suggest a physiological role for the competition of the two lipids for the N-PTB binding site. We propose that PtdIns(4,5)P₂ competes with sulfatides for binding to the N-PTB domain in order to mediate recycling of Dab2. Sulfatides drive Dab2 concentration on the surface of the activated platelet, but PtdIns(4,5)P2 drives the internalization of Dab2 for recycling into α-granules. Therefore, the two lipids balance the localization of Dab2 between the exterior and interior of the platelet. Sulfatide and PtdIns(4,5)P₂ competition for the N-PTB domain thus regulates Dab2's inhibitory role in aggregation.

We have demonstrated that the N-PTB domain specifically binds to sulfatides in a conserved site close by the one for PtdIns(4,5)P2 ligation. While the lipid binding sites overlap through Lys53, they are distinct from each other. Furthermore, the PtdIns(4,5)P₂ competes with sulfatides for PTB binding. In platelets, sulfatide binding mediates membrane localization of Dab2 upon platelet activation. Whereas sulfatides may stabilize Dab2 during platelet activation and compete with the αIIbβ3 integrin receptor for Dab2 ligation, PtdIns(4,5)P₂ participates in the internalization of the protein for its recycle and storage in α-granules. While each lipid drives a different localization of Dab2, the balanced competition between the two lipids plays a key role in platelet aggregation by regulating Dab2, an inhibitor of the αIIbβ3 integrin receptor. Perturbation of the role of sulfatides shifts the localization of Dab2 away from the surface of the platelet membrane, increasing Dab2-integrin receptor binding and ultimately decreasing platelet adhesion to fibrinogen. Disruption of PtdIns(4,5)P₂ binding, on the other hand, prevents the localization of Dab2 to α-granules in preparation for secondary aggregation. In summary, we have defined and characterized Dab2 N-PTB interaction with sulfatides. We have also determined the role of sulfatide and PtdIns(4,5)P₂ binding in the context of platelet aggregation. Furthermore, our findings suggest a physiological role for the competition between the two lipids, elucidating the mechanisms governing the localization of Dab2 during thrombosis. In addition, this work further defines integrin receptor regulation in the interest of therapeutic developments that specifically target the active conformation of the αIIβ3 integrin receptor using innate cellular mechanisms.

### Example 9

### Sulfatide Binding to P-Selectin Triggers De-Granulation of ADP Stimulated Platelets

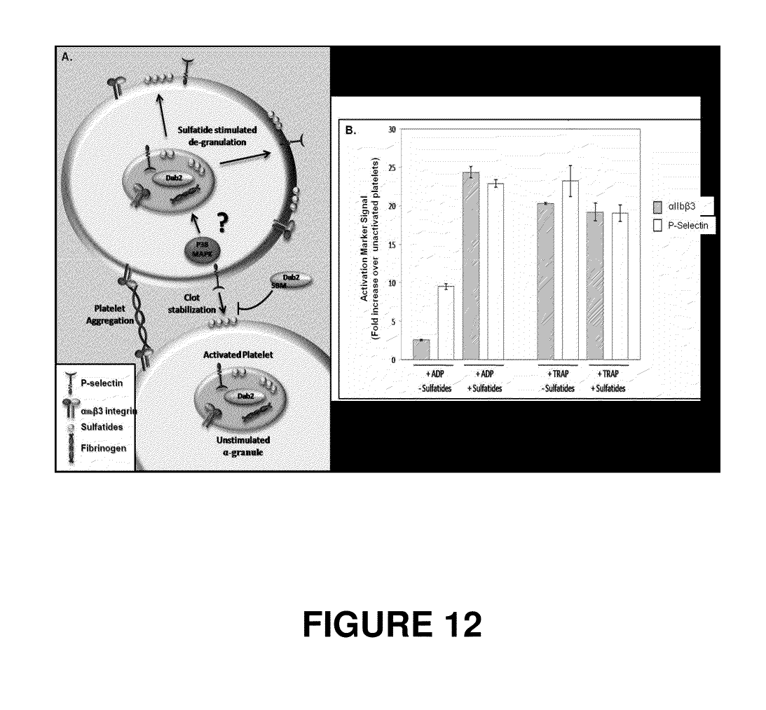

Resting platelets do not expiess P-selectin on their surface, however after stimulation with an agonist and subsequent de-granulation P-selectin becomes present on the platelet surface. Once present, P-selectin is able to interact with sulfatides which triggers an internal phosphorylation pathway thought to be mediated by p38 (FIG. 12A) (Merten 2005). This cascade results in further de-granulation and an increase in surface P-selectin (Merten 2005). To monitor the de-granulation of platelets, two activation markers were evaluated using fluorescent antibodies specific for P-selectin and the α_{IIb}β₃ integrin. Marker surface expression was quantified using fluorescence-activated cell sorting (FACS) analysis, and measuring the median fluorescent signal of 10,000 platelets. The median signal was used as our reference to quantify the data set as median values are less affected by the wide range of platelet signals and is therefore more representative of the population. When platelets were stimulated with ADP (30 μM) they show increases in both surface P-selecting and α_{IIb}β₃ integrin expression. In the presence of sulfatides (50 μg/mL), in the form of enriched liposomes, the levels of P-selectin and α_{IIb}β₃ integrin were further increased to about 20-25 fold their un-stimulated levels (FIG. 12B). A stronger agonist TRAP (10 μM) however was able to induce complete de-granulation, and the addition of sulfatides to TRAP stimulated platelets showed no affect on either P-selectin or α_{IIb}β₃ integrin levels (FIG. 12B). The levels of P-selectin and α_{IIb}β₃ integrin were measured among several different individuals and the levels varied greatly between them; however; consistently, sulfatides induced a ˜20 fold increase over un-stimulated platelets and a ˜10 fold increase over ADP stimulated platelets.

### Example 10

### N-PTB Sulfatide Binding Competes with P-Selectin and Inhibits Surface Expression of P-Selectin and αIIbβ3 Integrin

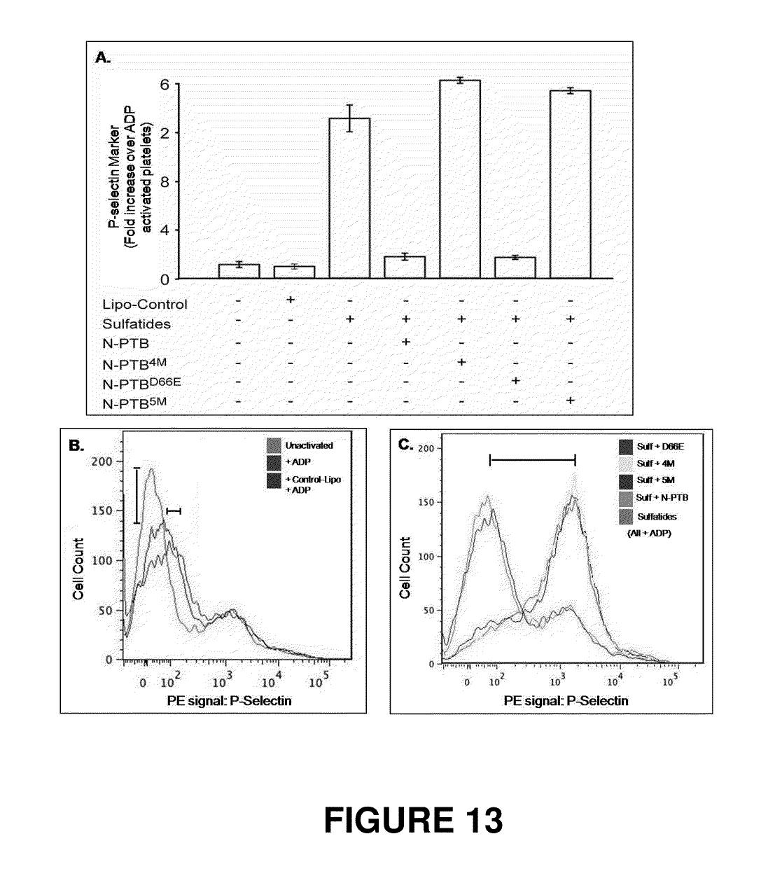

During platelet activation Dab2 is released from α-granules and is able to bind to either the α_{IIb}β₃ integrin or sulfatides. To define the role of Dab2 in the inhibition of sulfatide activation of platelets we monitored platelet surface P-selectin and α_{IIb}β₃ integrin in the presence of sulfatides and N-PTB wild type and mutants (FIG. 13). Washed platelets were monitored for their levels of surface P-selectin using R-phycoerythrin (PE) labeled-anti-human P-selectin quantified using the median fluorescent signal detected by FACS (FIG. 13A). Un-treated washed platelets contain two populations, defined by the FACS chromatogram, a peak at 10¹ PE signal defines low activation level while a second peak at 10³ represents highly activated platelets (FIG. 13B). The population of highly activated platelets in the untreated population most likely results from activation triggered by the washing process. ADP stimulation of the platelets results in a shift and broadening of the peak corresponding to low activated platelets, resulting in an increased median fluorescence (FIG. 13A-B). The addition of control liposomes, liposomes that do not contain sulfatides, to ADP stimulated platelets did not trigger any additional shift in the peak and the median fluorescence remained consistent with ADP treatment (FIG. 13A-B).

Upon the addition of sulfatides to ADP stimulated platelets there was a complete shift of the low activation peak to the highly activated population (FIG. 13C). This shift resulted in a roughly 12-fold increase in the median fluorescent signal compared to ADP treated platelets (FIG. 13A). The addition of N-PTB to the washed platelets was able to out-compete the P-selectin for sulfatide binding, and therefore, preventing the activation of the platelets. The low activation population was unaffected in the presence of N-PTB and the median fluorescent signal remained at ADP stimulated levels (FIG. 13A-C).

To determine the role of α_{IIb}β₃ integrin binding ability of N-PTB, sulfatide inhibition was measured using mutant N-PTB. N-PTB^{4M} is unable to bind to sulfatides, but inhibits fibrinogen binding to the α_{IIb}β₃ integrin. However, N-PTB^{4M} showed no inhibition of sulfatide induced activation (FIG. 13A-C). N-PTB^{D66E} is unable to bind to the α_{IIb}β₃ integrin but is still able to interact with sulfatides, and therefore showed complete inhibition of sulfatide induced activation (FIG. 13A-C). PTB^{5M}, N-PTB containing both the 4M and D66E mutations, cannot interact with either sulfatides or the α_{IIb}β₃ integrin and showed no affect on sulfatide induced activation (FIG. 13A-C).

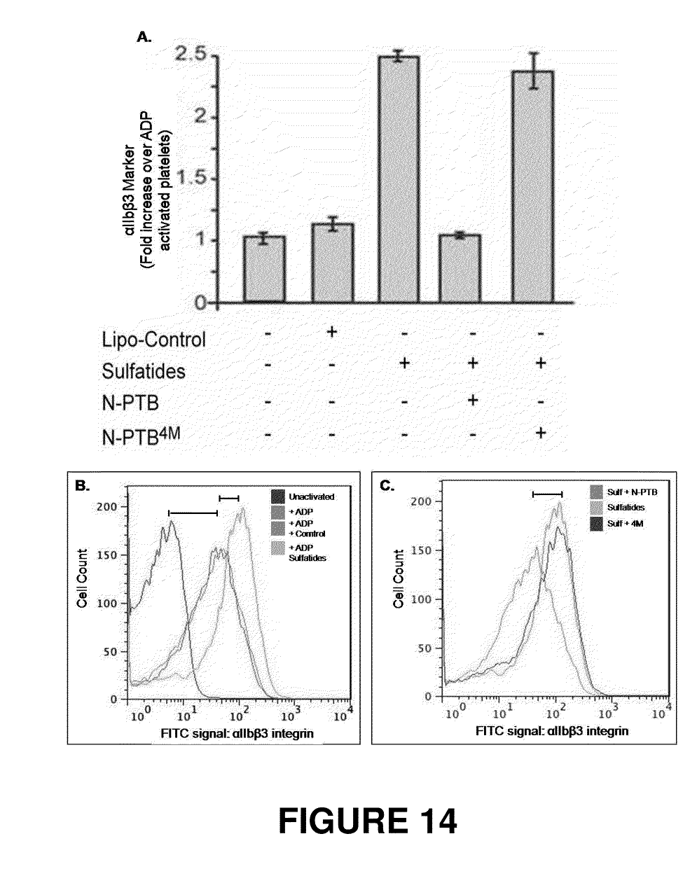

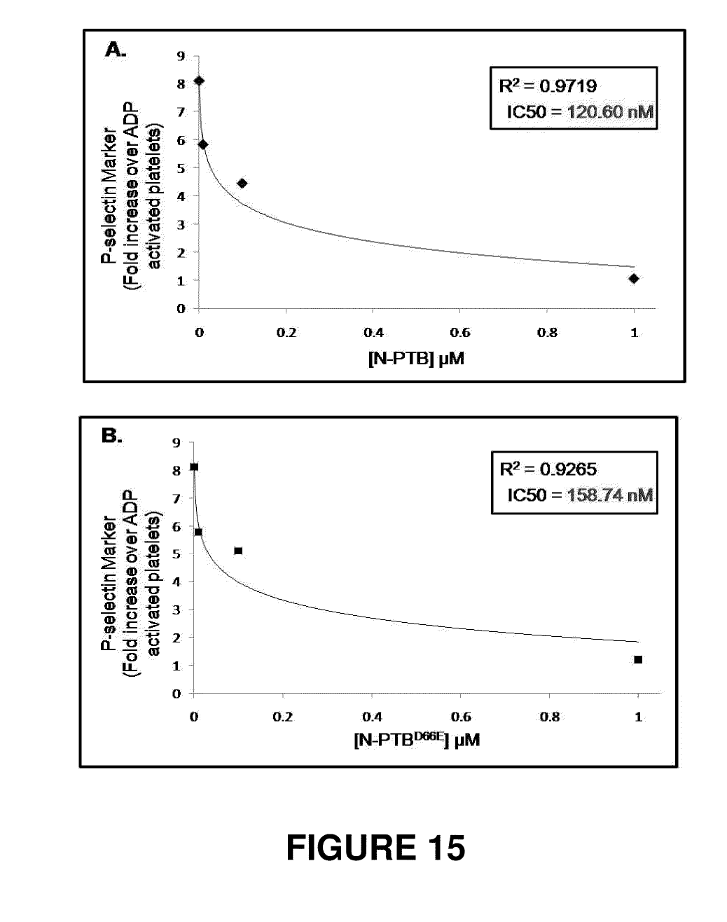

Sulfatide induced activation also increases surface α_{IIb}β₃ integrin through α-granule de-granulation. Surface α_{IIb}β₃ integrin was monitored using FITC-anti-human α_{IIb}β₃ integrin, and quantified using median fluorescence signal detected by flow cytometry. Resting platelets showed a peak of fluorescence below 10¹, the addition of ADP shifted the peak to above 10² (FIG. 14B). The addition of ADP stimulated not only the release of the α_{IIb}β₃ integrin but also the activation of the α_{IIb}β₃ integrin that is constitutively expressed on the surface, leading to a stronger response than the ADP effect on P-selectin. The addition of sulfatides further increased the fluorescence peak and caused a 2.5 fold increase in the median fluorescence (FIG. 14A-C). N-PTB inhibition of sulfatide induced expression of α_{IIb}β₃ integrin was dependent upon sulfatide binding, as N-PTB^{4M} showed no inhibition (FIG. 14A-C).

### Example 11

### N-PTB Binding of αIIbβ3 Integrin Doesn't Influence Sulfatide Inhibition

Dab2 splits into two pools during platelet aggregation, α_{IIb}β₃ integrin bound and sulfatide bound. When sulfatide binding is abolished an increase in α_{IIb}β₃ integrin binding is seen. To determine if N-PTB inhibition of sulfatide activation is increased with the abolition of α_{IIb}β₃ integrin binding, titrations of both N-PTB and N-PTB^{D66E} and an IC50 value was calculated (FIG. 15).

The titration curves matched a logarithmic model with a correlation greater than 0.9 for both curves. The calculated 1050 values were 120.60 nM for N-PTB (FIG. 15A), and 158.74 nM for N-PTB^{D66E} (FIG. 15B). These values indicate that when the α_{IIb}β₃ integrin bound pool of N-PTB shifts to become available for sulfatide binding the inhibitory affect of Dab2 is not changed. We surmise that this may be because the integrin bound pool is significantly smaller than the sulfatide pool, and thus the shift doesn't significantly affect sulfatide binding.

### Example 12

### N-PTB is Able to Prevent Sulfatide Induced Platelet Aggregation Under Flow

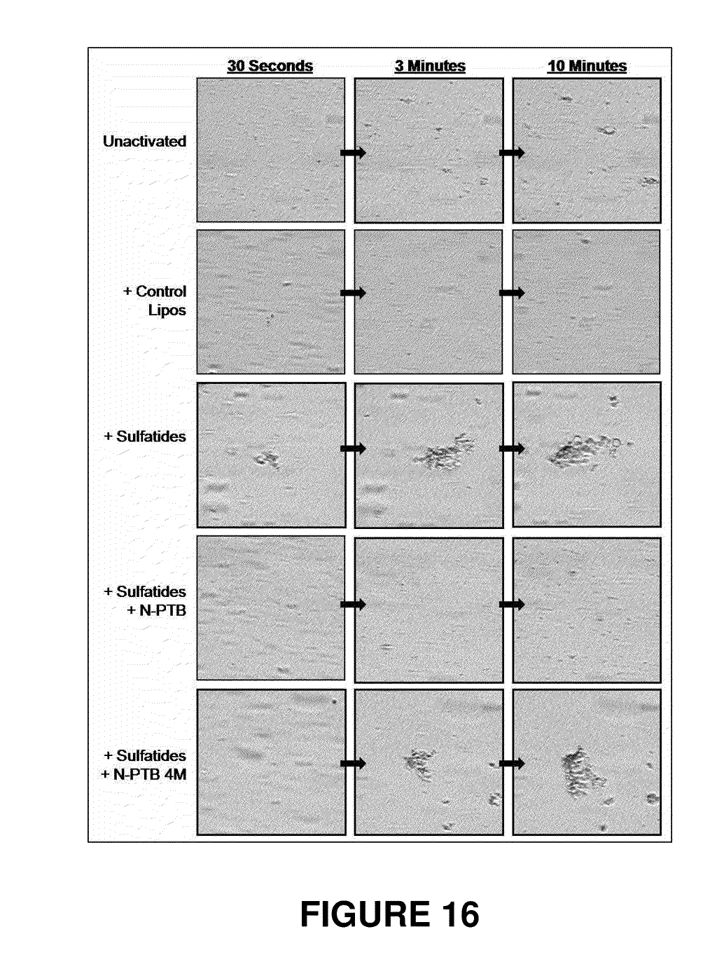

Platelet aggregation is mediated by α_{IIb}β₃ integrin interactions with fibrinogen and fibrin linking platelets together (Furie et al., J. Clin. Invest. 2005; 115 (12): 3355-3362; and Andrews et al., Thromb Res. 2004; 114:447-453). P-selectin links aggregated platelets together stabilizing clot formation (Merten 2001). Sulfatides stimulate increased levels of both α_{IIb}β₃ integrin and P-selectin expression on platelets (Merten 2005). To observe platelet aggregation under flow conditions, platelets were flowed through a microfluidics channel with a constant velocity and observed using light microscopy. The channel dimensions are 30×0.5×0.05 mm (length×width×height). Washed platelets were flowed at a shear rate of 70 s⁻¹, which is within the physiological range of capillary blood flow, through a channel coated with adhesive protein. The channel was coated by the incubation of human plasma, which contains soluble vWF, fibrinogen, and other adhesive proteins, in the channel for 2 hours allowing for the protein to adhere to the glass slide. Platelets would become activated by interacting with the adhesive proteins, and platelet-platelet interactions facilitated aggregate growth. Platelets were not previously stimulated with ADP, as we would allow for physiological activation of the platelets through interactions with the channel's adhesive proteins. Platelets, untreated or treated with sulfatide or control liposomes, were flowed through the channel for 10 minutes; and pictures were taken at 0 minutes, 3 minutes, and 10 minutes, based on our pervious observations of Dab2 functioning in platelet aggregation. Untreated and control liposome treated platelets showed activation and adhesion based on our observations of platelet monolayer formation, and low levels of aggregate formation (FIG. 16). Sulfatide liposomes stimulated much larger platelet aggregates than both untreated and control liposomes, observed qualitatively and quantified (FIG. 16). N-PTB was added along with sulfatide liposomes in order to constrain sulfatide induced aggregation, and with 10 μM N-PTB the platelet aggregates returned to the size of untreated platelets (FIG. 16). N-PTB^{4M} at 10 μM showed limited inhibition of platelet aggregate size but not adhesion. We surmise this is due to the increased binding to the α_{IIb}β₃ integrin which limits platelet-platelet aggregation but not adhesion.

### Example 13

### N-PTB Sulfatide Binding Limits Platelet-Leukocyte Interactions

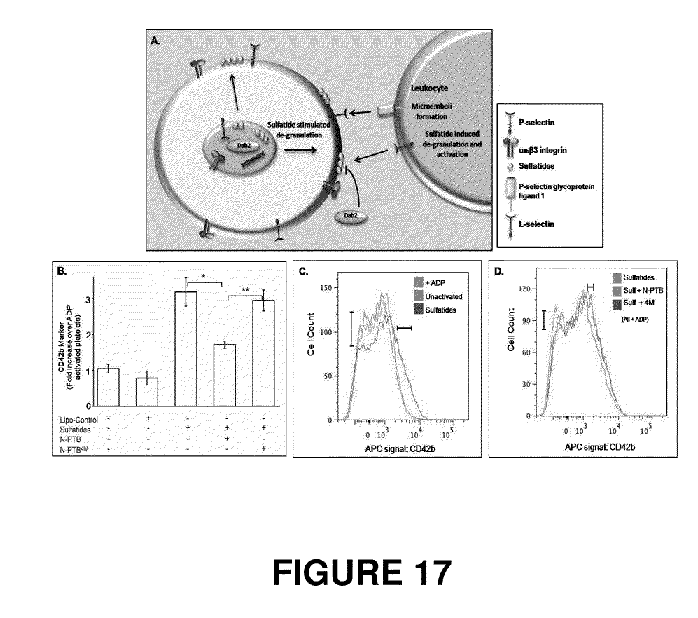

Platelet P-selectin mediates platelet-leukocyte interactions through binding of leukocyte PSGL-1 (Merten 2005). Sulfatide stimulation of platelets results in increased levels of P-selectin expression, and N-PTB is able to inhibit this stimulation (FIG. 17A). Sulfatides are also able to activate leukocytes directly through binding to L-selectin and an L-selectin independent pathway (FIG. 17A) (Ding et al., Journal of Leukocyte Biology. 2000; 68: 65-72). To determine sulfatide and N-PTB roles in platelet-leukocyte interactions, platelet and leukocyte mixtures (108 platelets/mL, 107 leukocytes/mL) were activated with ADP (30 μM) and incubated with control or sulfatide liposomes (50 μg/mL). Platelet binding of leukocytes was quantified through the fluorescence of allophycocyanin (APC) labeled anti-human CD42b (platelet marker) detected on leukocytes. The fluorescence was detected using flow cytometry analysis of platelet-leukocyte mixtures.

Platelet stimulation with ADP led to a very slight increase in platelet-leukocyte interactions corresponding to a slight increase in P-selectin (FIG. 17C). Addition of sulfatides resulted in a 3 fold increase in platelet-leukocyte interactions (FIG. 17B), with the flow cytometry chromatogram showing a positive shift in the fluorescence (FIG. 17C). The addition of N-PTB decreased platelet binding to leukocytes, seen by a decrease in median fluorescence signal as well as a negative shift of the fluorescence peak (FIG. 17A-D). N-PTB^{4M} showed no inhibition of platelet-leukocyte binding.

### Example 14

### N-PTB Abates Platelet Leukocyte Binding Under Flow Conditions

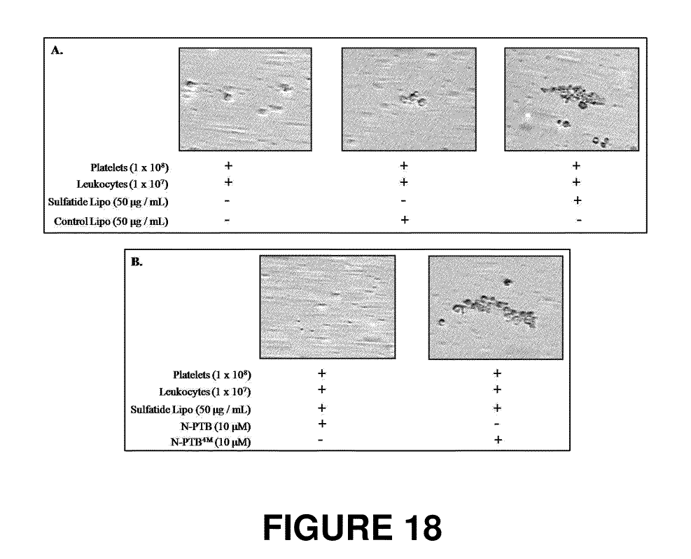

To quantify the ability of N-PTB to block platelet-leukocyte binding under physiological flow conditions, platelet and leukocyte mixtures were flown through a microfluidics device, as described above, and aggregation was measured using light microscopy. Untreated cell suspensions (108 platelets/mL, 107 leukocytes/mL) were flown for 10 minutes and the channel was scanned for leukocyte adhesion and inclusion into platelet aggregates and representative pictures were taken (FIG. 18A-B). The addition of sulfatides showed a marked increase in adhesive leukocytes and platelet-leukocyte clot size over untreated and control liposome treated samples (FIG. 18A).

When N-PTB (10 μM) was added, there was a great reduction in observed leukocyte adhesion as well as platelet-leukocyte aggregates (FIG. 18B). N-PTB^{4M} was unable to affect the adhesion and aggregation of leukocytes.

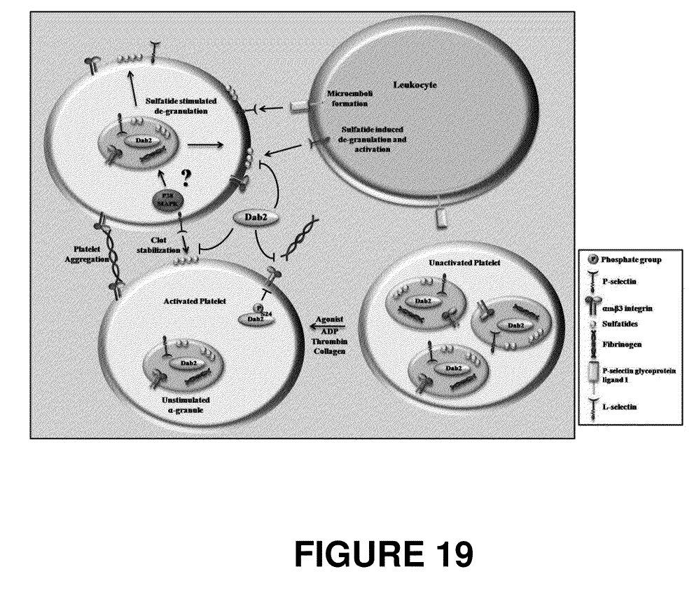

Overall, the release of Dab2 extracellularly inhibits platelet homotypic and heterotypic aggregation through a dual mechanism, one pool binds to surface α_{IIb}β₃ integrin inhibiting platelet-platelet interactions; another pool binds to sulfatides inhibiting secondary activation that is responsible for clot stability and leukocyte recruitment (FIG. 19).

Although certain presently preferred embodiments of the invention have been specifically described herein, it will be apparent to those skilled in the art to which the invention pertains that variations and modifications of the various embodiments shown and described herein may be made without departing from the spirit and scope of the invention. Accordingly, it is intended that the invention be limited only to the extent required by the appended claims and the applicable rules of law.

What is claimed is:

1. An isolated polypeptide comprising an amino acid sequence that binds sulfatides.

2. The polypeptide of claim 1, wherein the amino acid sequence contains amino acids 24-31 of SEQ ID NO: 1, amino acids 49-54 of SEQ. ID NO: 1, or modifications thereof that bind sulfatides.

3. The polypeptide of claim 1, wherein the amino acid sequence has the structure of Formula I
[D₁-L₁-D₂]ₙ (Formula I)
where D₁ and D₂ can be the same or different and denote sulfatide binding domains, L₁ denotes a linker, and n is an integer greater than 1.

4. The polypeptide of claim 3, wherein D₁ contains amino acids 24-31 of SEQ ID NO: 1, SEQ ID NO: 2, or SEQ ID NO: 3.

5. The polypeptide of claim 3, wherein D₂ contains amino acids 49-54 of SEQ ID NO: 1.

6. The polypeptide of claim 3, wherein L₁ comprises amino acids 32-48 of SEQ. ID NO: 1 or those containing a mutant form of the putative thrombin cleavage site within amino acis 32-48 of SEQ ID NO: 1.

7. The polypeptide of claim 1, comprising the amino acid sequence of SEQ ID NO: 1.

8. A method for inhibiting platelet aggregation comprising the step of contacting the polypeptide of claim 1 with platelets.

9. The method of claim 8, wherein the polypeptide contains amino acids 24-31 of SEQ ID NO: 1, amino acids 49-54 of SEQ. ID NO: 1, or modifications thereof that bind sulfatides.

10. The method of claim 8, wherein the polypeptide has the structure of Formula I
[D₁-L₁-D₂]ₙ (Formula I)
where D₁ and D₂ can be the same or different and denote sulfatide binding domains, L₁ denotes a linker, and n is an integer greater than 1.

11. The method of claim 10, wherein D₁ contains amino acids 24-31 of SEQ ID NO: 1, SEQ ID NO: 2, or SEQ ID NO: 3.

12. The method of claim 10, wherein D₂ contains amino acids 49-54 of SEQ ID NO: 1.

13. The method of claim 10, wherein L₁ comprises amino acids 32-48 of SEQ. ID NO: 1 or those containing a mutant form of the putative thrombin cleavage site within amino acis 32-48 of SEQ ID NO: 1.

14. The method of claim 8, comprising the amino acid sequence of SEQ ID NO: 1.

15. A method for promoting platelet aggregation comprising the step of contacting sulfatides with platelets.

16. The method of claim 15, wherein the platelets are activated.

17. A pharmaceutical composition comprising the polypeptide of claim 1.

18. An isolated nucleic acid molecule encoding for the polypeptide of claim 1.

19. A cell comprising a vector having the nucleic acid molecule of claim 19.

20. The cell of claim 19, wherein the cell is E. coli.
